(19) United States
(12) Patent Application Publication
(10) Pub. No.: US 2008/0200582 A1
(43) Pub. Date: Aug. 21, 2008

(54) High refractive index monomers, compositions and uses thereof

(76) Inventors: Liliana Craciun, Carmel, NY (US); Orest Polishchuk, Bayonne, NJ (US); George William Schriver, Fort Lee, NJ (US); Rudiger Hainz, Binzen (DE)

Correspondence Address: JoAnn Villamizar, Patent Department;Ciba Specialty Chemicals Corporation, 540 White Plains Road, P.O. Box 2005, Tarrytown, NY, 10591-9005, US

(21) Appl. No.: 12/070,183

(22) Filed: Feb. 14, 2008

Related U.S. Application Data
(60) Provisional application No. 60/997,942, filed on Oct. 5, 2007.
(60) Provisional application No. 60/902,530, filed on Feb. 20, 2007.

(51) Int. Cl. C08J 3/28; C08G 63/688

(52) U.S. Cl. 522/166; 525/451

(57) ABSTRACT

The invention relates to novel sulfur-containing (meth)acrylic monomers and compositions thereof characterized by a high refractive index, for optical and industrial applications. The invention also relates to a method for preparing high refractive index polymeric materials and more specifically to a method for formation of ultraviolet cast optical lenses and compositions thereof comprising the sulfur-containing (meth)acrylic monomers.

This application claims the benefit under 35 U.S.C. 119(e) of U.S. Provisional Application No. 60/997,942 filed on Oct. 5, 2007 which takes the benefit of Provisional Application No. 60/902,530, filed on Feb. 20, 2007 both herein incorporated entirely by reference.

### FIELD OF THE INVENTION

The invention relates to novel (meth)acrylic monomers and compositions thereof characterized by a high refractive index, for optical and industrial applications. The invention also relates to a method for preparing high refractive index polymeric materials and more specifically to a method and compositions for formation of ultraviolet cast optical lenses.

### BACKGROUND OF THE INVENTION

High refractive index (RI) materials are known for use in cast or coated products such as ophthalmic lenses, camera lenses, visors, safety glasses, watch glasses, video discs, monitors, displays, telecommunications systems, and medical/analytical equipment. In coating or film applications the high RI materials impart antireflective properties, brightness and gloss retention. In telecommunications, high RI monomers are especially suited for graded-index optical cables with superior performance in multi-mode fibers.

High refractive index materials work by enabling light to pass through the materials more quickly and are generally characterized by having reduced thickness for the same focusing power relative to those compositions without high RI materials. Particularly, ophthalmic lenses made from materials with a RI higher than conventional plastic (RI>1.5) are generally lighter because they require less material.

Polycarbonate plastic (PC) was introduced in the early 1980s as the first high-index plastic (RI 1.58) for lenses with increased impact resistance. However, PC lenses have poor optical qualities such as high birefringence and chromatic dispersion, they scratch easily and fuse in processing such as cutting and grinding. Polystyrenes are typically characterized by relatively high RI, but show increased optical dispersion combined with poor heat resistance. Polyurethanes have good impact resistance but poor weatherability, and are difficult to tint. Polysulfones have a high refractive index but are typically colored and difficult to process. While offering advantages over glass such as reduced weight and increased impact resistance, plastics still have shortcomings in their properties. There continues to be a need for new materials for making thinner, lighter and more resistant transparent optical materials.

Current high RI plastics include polyurethanes, polyesters, epoxy and episulfide resins. Most of the high RI plastics use thiourethane and episulfide chemistries with highly polarizable chemical moieties such as aromatics and sulfur. However, lenses produced from these materials suffer from after-cure yellowing and strong odors released during lens processing. In addition, these monomers have inherently long production cycles due to prolonged curing times needed for maintaining optical homogeneity. There is, therefore, a need for monomers which offer fast cure, high RI, low color, and low odor when cured or upon cutting and grinding, while maintaining optical homogeneity.

In particular, ultraviolet (UV)-casting or UV-cure manufacturing of optical lenses, a relatively new process for making optical lenses, presents challenging problems for high RI materials. Current high index monomers are neither appropriate for UV-cure manufacturing or do not have the quality adequate for ophthalmic lens applications. Thus development of innovative high RI monomers for UV-cured lenses is highly desirable.

U.S. Pat. Nos. 6,419,873, 6,557,734, 6,964,479 and 7,079,920 describe systems for UV-casting of plastic optical lenses herein entirely incorporated by reference.

(Meth)acrylate monomers are well known to those skilled in the art of UV-curing. They have excellent optical clarity and can be rapidly UV-cured via radical polymerization.

It is well known in the art that sulfur-containing (meth)acrylate monomers raise the refractive index in the formed polymer making up transparent optical material or lenses.

For example, U.S. Pat. Nos. 4,990,653 and 5,880,170, herein incorporated entirely by reference, and Japanese Application Nos. JP1993303003 and JP07206944 disclose sulfur-containing acrylic compositions giving a cured product useful in lenses.

The present invention includes novel high RI (meth)acrylate monomers and compositions that exhibit a RI of 1.58 or more, preferably 1.60 or more.

The present invention also includes optical materials which in addition to comprising sulfur containing (meth)acrylates also include organic-inorganic hybrid materials.

Organic-inorganic hybrid materials in combination with specific sulfur containing (meth)acrylates are known for use in optical coatings and disclosed in U.S. Publication Application Nos. 2006/0147674, 2006/0147703 and 2006/147702 herein entirely incorporated by reference.

PCT Application No. 2006/065660 discloses metal containing compositions formed from ethylenically unsaturated groups containing a metal and a prepolymer herein incorporated entirely by reference.

Additionally, Japanese Application No. JP2005314661 discloses a plastic solid containing polyfunctional sulfur-containing methacrylate monomers in combination with TiO₂.

Japanese Application No. JP1996157320 discloses metal oxides in combination with sulfur containing methacrylates for use in dental materials.

Monomers functionalized with groups which have the ability to chelate or bridge metals can be combined with the high RI monomers of the invention. This combination gives improved high RI homogeneous polymer composite materials. High RI monomers may be advantageous in these organic-inorganic hybrid materials as they may provide a better RI match to the inorganic component giving improved clarity and reduced haze.

### SUMMARY OF THE INVENTION

The invention encompasses several compositional embodiments.

### Generalized Classes of High Refractive Index Monomers

The invention encompasses high refractive index (RI) monomers selected from the group consisting of the formulae (1), (2), (3) and mixtures thereof:

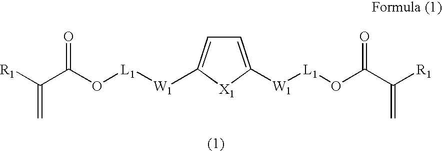

where
L₁ is defined as C₁-C₈ alkylene optionally interrupted by —S—, —SO₂—, —SO— and/or oxygen,
W₁ is a bond, sulfur or oxygen,
with the proviso that at least one of -L₁-W₁— or —W₁-L₁- contains at least one —S—, —SO₂— or —SO—,

### X₁ is S, SO or SO₂,

and
R₁ is independently H or CH₃,

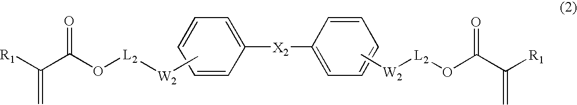

where
X₂ is a divalent linking group defined as a bond, —SO₂—, —SO—, —S—, —C(CH₃)₂—, —(CH₂)ₙ—S—(CH₂)ₙ—, —(CH₂)ₙ—SO—(CH₂)ₙ—, —(CH₂)ₙ—SO₂—(CH₂)ₙ—, —S—(CH₂)ₙ—S—, —SO—(CH₂)ₙ—SO— or —SO₂—(CH₂)ₙ—SO₂—,
and
W₂ is defined as a bond, sulfur, oxygen or a divalent linking group selected from the group consisting of —CONR₃—, —NR₃CO—, —SCONR₃—, —R₃NOCS—, —NR₃COS—, —SOCNR₃—, —CSO—, —OSC—, —COS—, —SOC—, —CSS—, —SSC—, —OCO—, —COO—, —SCOO—, —OOCS—, —OCONR₃— and —R₃NOCO—,
L₂ is C₁-C₁₀ alkylene which is optionally interrupted by W₂, —S—, —SO₂—, —SO— or oxygen,
with the proviso that at least one of -L₂-W₂— or —W₂-L₂- contains at least one of the divalent linking groups selected from the group consisting of —SCONR₃—, —R₃NOCS—, —NR₃COS—, —SOCNR₃—, —CSO—, —OSC—, —COS—, —SOC—, —CSS—, —SSC—, —SCOO—, and —OOCS—,
or
at least one of -L₂-W₂— or —W₂-L₂- contains —CONR₃—, —R₃NCO—, —OCONR₃—, —R₃NOCO—, —OCO—, —COO—, and at least one —S—, —SO₂— or —SO—,
or
-L₂-W₂— or —W₂-L₂- is a branched or linear C₁-C₄ alkylene substituted by OR₄ or SR₄,
R₃ is defined independently as H or CH₃,
R₄ is C₁-C₄ branched or linear alkyl or substituted or unsubstituted phenyl
and
R₁ is defined independently as H or CH₃;

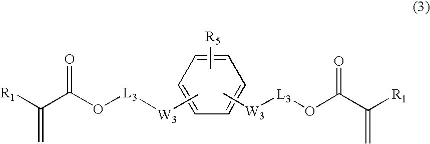

wherein
W₃ is a bond, sulfur, oxygen or a divalent linking group selected from the group consisting of —CONR₃—, —NR₃CO—, —SCONR₃—, —R₃NOCS—, —NR₃COS—, —SOCNR₃—, —CSO—, —OSC—, —COS—, —SOC—, —CSS—, —SSC—, —OCO—, —COO—, —SCOO—, —OOCS—, —OCONR₃—, and —R₃NOCO—,
L₃ is C₁-C₁₀ alkylene which is optionally interrupted by W₃, —S—, —SO₂—, —SO— and/or oxygen,
with the proviso that at least one of -L₃-W₃— or —W₃-L₃- must contain at least one of the divalent linking groups selected from the group consisting of —SCONR₃—, —R₃NOCS—, —NR₃COS—, —SOCNR₃—, —CSO—, —OSC—, —COS—, —SOC—, —CSS—, —SSC—, —SCOO—, and —OOCS—, or
at least one of -L₃-W₃— or —W₃-L₃- contain —CONR₃—, —R₃NCO—, —OCONR₃—, —R₃NOCO—, —OCO—, —COO—, and at least one —S—, —SO₂— or —SO—,
or
—W₃-L₃- or -L₃-W₃— is branched or linear C₁-C₄ alkylene substituted by OR₄ or SR₄,
R₃ is defined independently as H or CH₃,
R₄ is branched or linear C₁-C₄alkyl or substituted or unsubstituted phenyl,
R₁ is independently H or CH₃, and
R₅ is H or branched or linear C₁-C₄ alkyl.

### Specific Novel High Refractive Index Monomers

The invention also embodies a number of specific high refractive index (RI) monomers or mixtures thereof which are believed by the inventors to be novel.

These include the specific monomers listed in the Table 1 below:

TABLE 1
Specific High RI Monomers

| | |
|---|---|
| | 1 |
| 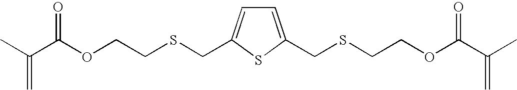 | |
| | 2 |
| 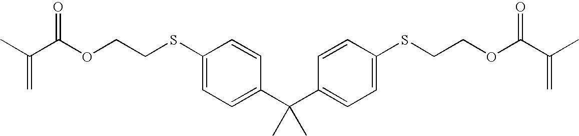 | |
| | 3 |
| 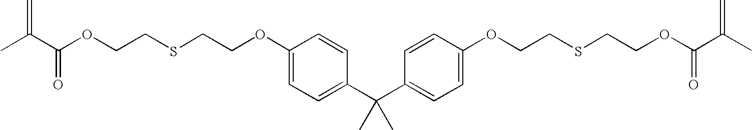 | |
| | 4 |
| 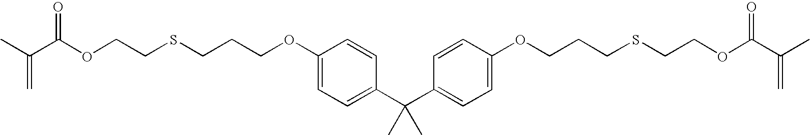 | |
| | 6 |
| 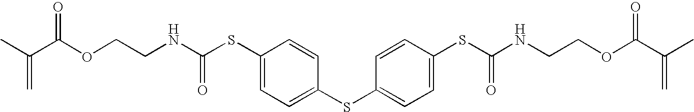 | |
| | 7 |
| 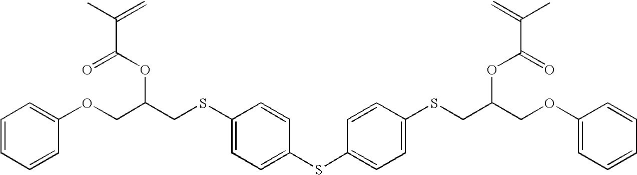 | |
| | 9 |
| 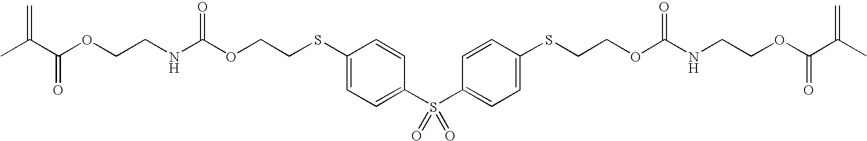 | |
| | 10 |
| 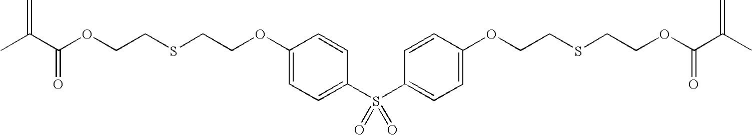 | |
| | 11 |
| 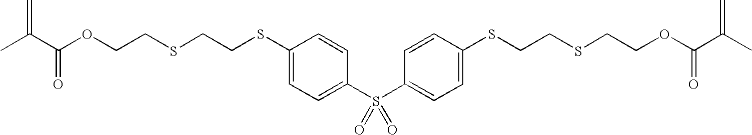 | |
| | 14 |
| 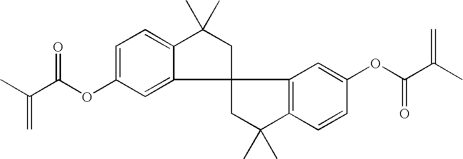 | |
| | 15 |
| 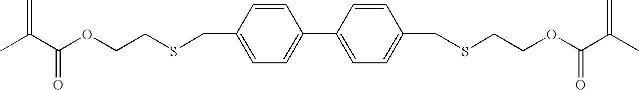 | |
| | 22 |
| 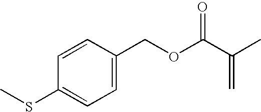 | |
| | 23 |
| 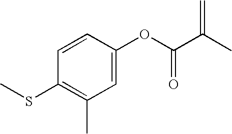 | |
| | 24 |
| 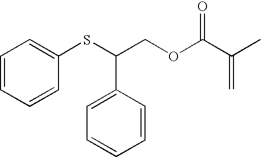 | |

### High Refractive Index Transparent Polymer Compositions

Furthermore the invention embodies a high refractive index transparent plastic composition comprising a plastic formed from at least one of the monomers selected from the group consisting of formulae (1), (2), (3) and mixtures thereof,

optionally, a functionalized or surface treated nanoparticle,
and
optionally, at least one monomer selected from the group consisting of mono(meth)acrylate aromatic sulfur-containing monomers.

### High Index of Refraction Ultraviolet-Cast (UV-Cast) Optical Lens

The invention also encompasses a UV-cast optical lens formed from at least one of the monomers selected from the group consisting of formulae (1), (4), (5) and mixtures thereof.

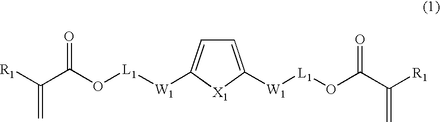

where
L₁ is defined as C₁-C₈ alkylene optionally interrupted by —S—, —SO₂—, —SO— and/or oxygen,
W₁ is a bond, sulfur or oxygen,
with the proviso that -L₁-W₁— or —W₁-L₁- contains at least one —S—, —SO₂— or —SO—,

### X₁ is S, SO or SO₂,

and
R₁ is independently H or CH₃;

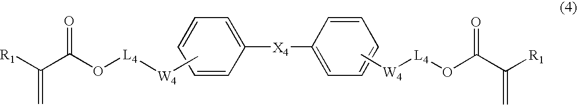

wherein
X₄ is a divalent linking group defined as a bond, —SO₂—, —SO—, —S—, —C(CH₃)₂—, —(CH₂)ₙ—S—(CH₂)ₙ—, —(CH₂)ₙ—SO—(CH₂)ₙ—, —(CH₂)ₙ—SO₂—(CH₂)ₙ—, —S—(CH₂)ₙ—S—, —SO—(CH₂)ₙ—SO— or —SO₂—(CH₂)ₙ—SO₂—,
n is 1-4,
W₄ is defined as a bond, sulfur, oxygen or a divalent linking group selected from the group consisting of —OCO—, —COO—, —CO—, —SO—, —SO₂—, —OCOO—, —OOCO—, —CONR₃—, —NR₃CO—, —SCONR₃—, —R₃NOCS—, —NR₃COS—, —SOCNR₃—, —CSO—, —OSC—, —COS—, —SOC—, —OCO—, —COO—, —CSS—, —SSC—, —SCOO—, —OOCS—, —OCONR₃— and —R₃NOCO—,
L₄ is C₁-C₁₀ alkylene which is optionally interrupted by oxygen, —S—, —SO₂—, —SO— or W₄,
or
L₄ is a branched or linear C₁-C₄ alkylene substituted by OH, OR₄ or SR₄,
R₃ is defined independently as H or CH₃,
R₄ is branched or linear C₁-C₄ alkyl or substituted or unsubstituted phenyl, and
R₁ is defined independently as H or CH₃,
and

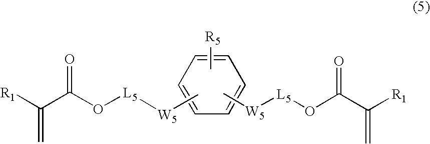

wherein
W₅ is a bond, oxygen of sulfur or a divalent linking group selected from the group consisting of —OCO—, —COO—, —CO—, —SO—, —SO₂—, —OCOO—, —OOCO—, —CONR₃—, —NR₃CO—, —SCONR₃—, —R₃NOCS, —NR₃COS—, —SOCNR₃—, —CSO—, —OSC—, —COS—, —SOC—, —OCO—, —COO—, —CSS—, —SSC—, —SCOO—, —OOCS—, —OCONR₃— and —R₃NOCO—,
L₅ is C₁-C₁₀ alkylene optionally interrupted by oxygen, —S—, —SO₂—, —SO— or W₅,
or L₅ is a branched or linear C₁-C₄ alkylene substituted by OH, OR₄ or SR₄,
R₃ is defined independently as hydrogen or CH₃,
R₄ is branched or linear C₁-C₄ alkyl or substituted or unsubstituted phenyl,
R₅ is hydrogen or branched or linear C₁-C₄ alkyl, and
R₁ is defined independently as H or CH₃,
with the proviso that at least one of the -L₅-W₅— or —W₅-L₅- contains at least one sulfur.

In addition to at least one of the monomers of formulae (1), (4), (5) or mixtures thereof, the UV-cast lens may optionally contain functionalized or surface treated nanoparticles, wherein the nanoparticles are an inorganic particle such as a metal, metal oxide, metal nitride, metal carbide, metal chloride or mixtures thereof.

The phrase “functionalized or surface treated nanoparticle” means that the nanoparticle is treated with organic surface modifying agents such as carboxylic acids, silanes and/or dispersants to help compatiblize the nanoparticle with a polymeric matrix.

The invention encompasses several method embodiments:

The first of which is

a method of forming a high refractive index transparent material wherein the transparent material is a polymeric molded body, coating or film and the method comprises the steps:
placing a liquid composition into a mold cavity or assembly, wherein the mold assembly or cavity comprises a front mold member and a back mold member,
or
spreading the liquid composition onto a substrate to form a film or coating,
the liquid composition comprises at least one monomer selected from the group consisting of
formula (1), (2) and (3),
optionally, a surface treated or functionalized nanoparticle,
and
a photoinitiator,
   and
   directing activating light toward at least one of the mold members, the film or coating to effect cure.

Secondly, a method of forming a high refractive index polymeric eyeglass lens comprising the steps:

placing a liquid lens forming composition in a mold cavity or a mold assembly, wherein the mold assembly comprises a front mold member and a back mold member, the lens forming composition comprises:
at least one monomer selected from the group consisting of formula (1), (4) and (5),
optionally, a surface treated or functionalized nanoparticle,
and
a photoinitiator;
   and
   directing activating light toward at least one of the mold members subsequent to initiating cure of the lens to form the eyeglass lens.

### DETAILED DESCRIPTION OF THE INVENTION

### A Generalized Class of High Refractive Index Monomers

The invention encompasses high refractive index (RI) monomers of the formulae (1), (2), (3) and mixtures thereof:

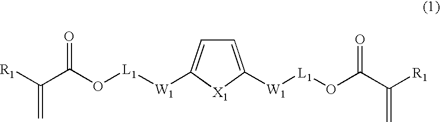

where
L₁ is defined as C₁-C₈ alkylene optionally interrupted by sulfur and/or oxygen,
W₁ is a bond, sulfur or oxygen,
with the proviso that -L₁-W₁— or —W₁-L₁- contain at least one —S—, —SO₂— or —SO—,

### X₁ is S, SO or SO₂, and

R₁ is independently H or CH₃,
L₁ is for example, —CH₂—CH₂—S—CH₂—, —CH₂—CH₂—S—CH₂—CH₂—, —CH₂—CH₂—S—, —CH₂—CH₂—O—CH₂—CH₂—S— and —CH₂—CH₂—O—CH₂—CH₂—S—CH₂—.

When sulfur interrupts a C₁-C₈ alkylene chain, the sulfur may be oxidized to SO or SO₂. Thus L₁ may independently be for example: —CH₂—CH₂—SO—CH₂—, —CH₂—CH₂—SO₂—CH₂—, —CH₂—CH₂—SO—CH₂—CH₂— and —CH₂—CH₂—SO₂—CH₂—CH₂—.

-L₁-W₁— or —W₁-L₁- for example may be —CH₂—CH₂—S—CH₂—, —CH₂—CH₂—S—, —S—CH₂—CH₂—, —O—CH₂—CH₂—S—CH₂— or —CH₂—S—CH₂—CH₂—O—,
C₁-C₈ alkylene is for example C₁-C₄ or C₁-C₆ alkylene.

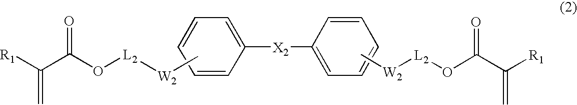

where
X₂ is a divalent linking group defined as a bond, —SO₂—, —SO—, —S—, —C(CH₃)₂—, —(CH₂)ₙ—S—(CH₂)ₙ—, —(CH₂)ₙ—SO—(CH₂)ₙ—, —(CH₂)ₙ—SO₂—(CH₂)ₙ—, —S—(CH₂)ₙ—S—, —SO—(CH₂)ₙ—SO— or —SO₂—(CH₂)ₙ—SO₂—,
and
W₂ is defined as a bond, sulfur, oxygen or a divalent linking group selected from the group consisting of —CONR₃—, —NR₃CO—, —SCONR₃—, —R₃NOCS—, —NR₃COS—, —SOCNR₃—, —CSO—, —OSC—, —COS—, —SOC—, —CSS—, —SSC—, —OCO—, —COO—, —SCOO—, —OOCS—, —OCONR₃— and —R₃NOCO—,
L₂ is C₁-C₁₀ alkylene which is optionally interrupted by W₂, —S—, —SO—, —O— and/or —SO₂—,
with the proviso that at least one of -L₂-W₂— or —W₂-L₂- contain at least one of the divalent linking groups selected from the group consisting of —SCONR₃—, —R₃NOCS—, —NR₃COS—, —SOCNR₃—, —CSO—, —OSC—, —COS—, —SOC—, —CSS—, —SSC—, —SCOO—, and —OOCS—,
or
at least one of -L₂-W₂— or —W₂-L₂- contain —CONR₃—, —R₃NCO—, —OCONR₃—, —R₃NOCO—, —OCO—, —COO—, and at least one —S—, —SO₂— or —SO—,
or
-L₂-W₂— or —W₂-L₂- is a branched or linear C₁-C₄ alkylene substituted by OR₄ or SR₄,
R₃ is defined independently as H or CH₃,
R₄ is C₁-C₄ branched or linear alkyl or substituted or unsubstituted phenyl, and
R₁ is independently H or CH₃.

For example, L₂ may be —CH₂—CH₂—S—CH₂—CH₂—O—CONH—CH₂—CH₂—, —CH₂—SOCNH—CH₂—CH₂—, —CH₂—SOCNH—CH₂—CH₂—O—CH₂—CH₂— or —CH₂—SCONH—CH₂—S—CH₂—CH₂—. -L₂-W₂— or —W₂-L₂- may be for example —CH₂—CH₂—S—CONH—CH₂—S— or —CH₂—CH₂—S—CONH—CH₂—.

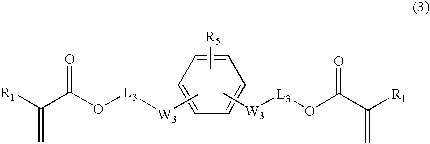

wherein
W₃ is a bond, sulfur, oxygen or a divalent linking group selected from the group consisting of —CONR₃—, —NR₃CO—, —SCONR₃—, —R₃NOCS—, —NR₃COS—, —SOCNR₃—, —CSO—, —OSC—, —COS—, —SOC—, —CSS—, —SSC—, —OCO—, —COO—, —SCOO—, —OOCS—, —OCONR₃— and —R₃NOCO—, L₃ is C₁-C₁₀ alkylene which is optionally interrupted by W₃, —S—, —SO—, —SO₂— and/or —O—,
with the proviso that at least one of -L₃-W₃— or —W₃-L₃- contain at least one of the divalent linking groups selected from the group consisting of —SCONR₃—, —R₃NOCS—, —NR₃COS—, —SOCNR₃—, —CSO—, —OSC—, —COS—, —SOC—, —CSS—, —SSC—, —SCOO—, and —OOCS—,
or
at least one of -L₃-W₃— or —W₃-L₃- contain —CONR₃—, —R₃NCO—, —OCONR₃—, —R₃NOCO—, —OCO—, —COO—, and at least one —S—, —SO₂— or —SO—,
or
—W₃-L₃- or -L₃-W₃— is branched or linear C₁-C₄ alkylene substituted by OR₄ or SR₄,
R₃ is defined independently as hydrogen or CH₃,
R₄ is branched or linear C₁-C₄ alkyl or substituted or unsubstituted phenyl,
R₅ is hydrogen or branched or linear C₁-C₄ alkyl, and
R₁ is independently H or CH₃.

Preferably R₄ is a substituted or unsubstituted phenyl.

L₃, for example may be —CH₂—CH₂—S—CH₂—CH₂—O—CONH—CH₂—CH₂—, —CH₂—SOCNH—CH₂CH₂—, —CH₂—SOCNH—CH₂—CH₂—O—CH₂—CH₂— or —CH₂—SCONH—CH₂—S—CH₂—CH₂—. -L₃-W₃— or —W₃-L₃- may be for example —CH₂—CH₂—S—CONH—CH₂—S— or —S—CH₂—HNOC—S—CH₂—CH₂—.

C₁-C₁₀ alkylene for purposes of the invention may be for example, C₁-C₂₃, C₁-C₄, C₁-C₆ or C₁-C₈.

Furthermore the invention embodies a transparent high refractive index plastic composition comprising a plastic formed from at least one of the formulae (1), (2), (3) or mixtures thereof,

optionally a surface treated or functionalized nanoparticle,
and
optionally at least one monomer selected from the group consisting of mono(meth)acrylate aromatic sulfur-containing monomers.

The preparation of formulae 1-5 may be formed by typical methods known in the art. For example, U.S. Pat. No. 3,824,293 discloses a method for the synthesis of bisthioethers herein incorporated entirely by reference. The bisthioethers may then be reacted with a (meth)acrylate to form the (meth)acrylates of formulae 1-5. U.S. Pat. No. 3,824,293 teaches to prepare bisthioethers by condensing an alkali metal salt of a hydroxyalkyl with an aromatic halogen compound.

As the novel monomers (formulae 1-5) are used in optical lenses or applications which require very little or no color, the intermediates used in preparation of the monomers are preferably also colorless and of high purity. Bisthioethers may serve as intermediates for formulae 1-5. It has surprisingly been discovered that the product of condensing an alkali metal salt of a hydroxyalkyl mercaptan with an aromatic halogen compound can be significantly improved by reacting a potassium metal salt of the hydroxyalkyl mercaptan with the aromatic halogen compound in a solvent derived from an amide.

Although the use of amide solvents has been recognized as a good solvent choice for potassium aryl thiolates (see Campbell, J. R. et al, J. Org. Chem., 1964, 29, 1830-1833), it is surprising that the presence of the hydroxy groups on the hydroxyalkyl mercaptans does not give appreciable side products.

Thus, the invention embodies:

A method of preparing bisthioethers of formulae (2′) and (3′)

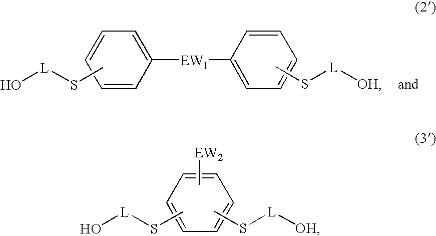

wherein L is C₂-C₆ alkyl or C₁-C₆ alkylene interrupted by oxygen or sulfur, and EW₁ and EW₂ are electron withdrawing groups,
by condensing a potassium salt of a hydroxyalkyl mercaptan with an aromatic halogen compound,
wherein the condensation takes place in a solvent selected from the group consisting of
dimethylformamide, dimethylacetamide, N,N-dimethylbutyramide, N,N-dibutylacetamide and N-methylpyrrolidinone.

The potassium salt of the hydroxyalkyl mercaptan is preferably formed from the reaction of the hydroxyakyl mercaptan with K₂CO₃.

The alkyl of the hydroxyalkyl mercaptan may be branched or unbranched C₁-C₆ alkyl, preferably branched or unbranched C₂-C₆ alkyl. Thus the hydroxy and mercaptan functionalities are aliphatic. The alkyl group may be further substituted with say an aromatic ring.

The mercaptan and hydroxy groups on the hydroxyalkyl mercaptan may be primary, secondary or tertiary. For examples, both the mercaptan and hydroxy groups may be on opposite terminal ends of the alkyl group such as in 2-hydroxyethyl mercaptan. Aromatic rings include benzene, fused benzene rings and thiophene.

C₁-C₆ alkylene, preferably C₂-C₆ alkylene optionally interrupted by oxygen or sulfur may be for example HO—CH₂CH₂—S—CH₂CH₂—SH, and HO—CH₂CH₂CH₂—O—CH₂CH₂CH₂—SH.

An aromatic halogen for purposes of the invention means halogen(s) directly substituted on the aromatic ring(s).

EW₁ may be for example —CX₂—, —SO—, —SO₂— and —C(═O)—. —CX₂— may be for example —CCl₂— and —CF₂—.

EW₂ may be for example —CX₃, —NO₂, —CN and —X. —CX₃ may be for example —CF₃ and —CCl₃.

The EW₂ substitution on formula (3′) may be 1 to 4.

Transparent for purposes of the invention means that the plastic composition has a greater than 90% transmittance of light in the 400-700 nm range. The compositions may for example have a transmittance of at least about 95% and more typically at least about 99%. The percent transmittance of the composition refers to the cured composition although the liquid before cure is frequently also characterized by high transparency and low color.

The sulfur-containing monomers forming the UV-cast optical lens should comprise the bulk of the lens. For example, at least about 75 to 100 wt. % of the sulfur-containing monomers, especially about 80 to about 99 wt. %, more especially about 85 to about 98 wt. %, make up the formed optical lens. The weight % of the sulfur-containing monomers is based on the total weight of the cured or formed UV-cast lens. Thus the pre-cured compositions may include solvents and the like which do not become part of the cast UV-lens. Therefore, the wt. % of the sulfur-containing monomers does not include solvents or components which do not become part of the cast lens after curing.

A typical composition includes up to 98% by weight of the high index monomers of the invention and up to 5% by weight of at least one photoinitiator effective to promote polymerization, with other optional components such as reactive diluents, crosslinkers, light stabilizers, mold-release agents, or dyes.

In the event that nanoparticles are included with the sulfur-containing monomers in a UV-cast lens, the sulfur-containing monomers should make up anywhere from about 95 wt. % to about 50 wt. %, especially 92 wt. % to about 55 wt. %, most especially about 90 wt. % to about 60 wt. % of the UV-cast composition, that is, based on the wt. % of the composition of the cast lens after curing.

### Nanoparticle Definition

Nanoparticles for purposes of the invention mean an average diameter up to and including about 200 nm. Preferable, the particle diameter is up to and including about 100 nm, more preferably up to and including about 70 nm diameter and most preferably in the range of about 5-50 nm. For examples, the majority of nanoparticles may be sized to have a volume average of about 5 nm to about 50 nm, about 5 nm to about 70 nm, about 5 nm to about 100 nm and about 5 nm to about 200 nm.

Majority is defined to be over 50% by weight of the nanoparticles, and more preferably from about 67 to 90% by weight. A minority of nanoparticles is defined to be less than 50% by weight of the nanoparticles, and more preferably from about 40 to 10% by weight.

A nanoparticle is generally an inorganic particle such as a metal, metal oxide, metal nitride, metal carbide or metal chloride. In accordance with the present invention, the use of high index nanoparticles increases the refractive index of compositions incorporating the same. High index nanoparticles such as zirconia, silica, titania, antimony, mixtures of metal oxides, mixed metal oxides, and mixtures thereof are acceptably envisioned.

The metal of inorganic nanoparticle may be Zr, Hf, Ge, Ti, Pb, Gd, Sn, Zn, Ni, Na, Li, K, Ce, Nb, Eu, In, Al, Fe, Mn, Nd, Cu, Sb, Mg, Ag and Y. If the nanoparticle is an elemental metal, Zr, Zn, Ti, Al and Ce are the most preferred. For example, the metal may be an elemental metal or a metal oxide such as, Zr, ZrO, ZrO₂, Ti, Ce, CeO₂ and TiO₂. Preferably, the nanoparticle comprises Ce, CeO₂, Zr, ZrO₂, Zn, ZnO₂, A1, Al₂O₃, Ti, TiO₂ or mixtures thereof.

The surface treated nanoparticles may make up about 5 to about 50 wt. % of the UV-cast lens. For example, the surface treated nanoparticles may make up about 8 to about 45 wt. %, about 10 to about 40 wt. % of the cured lens.

### Surface Modification or Functionalization of Nanoparticles

Surface-treating or functionalizing the nanoparticles can provide a stable dispersion in the polymeric resin. Preferably, the surface-treatment stabilizes the nanoparticles so that the particles will be well dispersed in the polymerizable resin and results in a substantially homogeneous composition. Furthermore, the nanoparticles can be modified over at least a portion of its surface with a surface treatment agent so that the stabilized particle can copolymerize or react with the polymerizable resin during curing.

See for example, PCT Publication No. WO 2006/065660 which discloses metal-containing compositions comprising a metal-containing precursor unit and a prepolymer unit with an initiator to induce polymerization. The metal-containing precursor unit contains a metal bound to ethylenically unsaturated moieties of the type listed below:

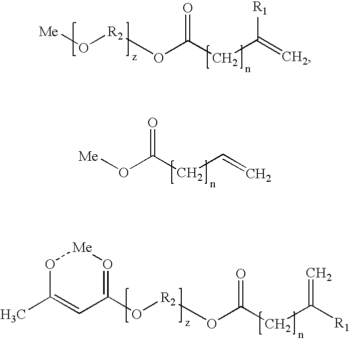

where
R₁ represents H atom, CH₃ or an alkyl group containing 2-8 carbon atoms, a group containing a halogen atom, or a hydroxyalkyl group; R₂ represents an alkyl group, C₁-C₆ alkylene or a substituted or unsubstituted aryl group; z is 1-3; n is 0-6, and Me represents metal.

Silica is compatible with inorganic oxides and thus may serve as a coupler between the two matrices in sol-gel processes for example. Thus silanes may be used as coupler or crosslinking agent, or as surface treatment agents for the inorganic phase.

In general, a surface treatment agent has a first end that will attach to the particle surface (covalently, ionically or through strong physical adsorption) and a second end that imparts compatibility of the particle with the resin and/or reacts with the resin during curing. Examples of surface treatment agents include: alcohols, amines, carboxylic acids, sulfonic acids, phosphonic acids, thiols, silanes and titanates. The preferred type of treatment agent is determined, in part, by the chemical nature of the metal oxide surface. Silanes are preferred for silica and other siliceous fillers. The surface-modification can be done either subsequent to mixing with the monomers or after mixing. When silanes are employed, reaction of the silanes with the particle or nanoparticle surface is preferred prior to incorporation into the resin. The required amount of surface modifier is dependent upon several factors such as particle size, particle type, modifier molecular weight, and modifier type. In general, it is preferred that about a monolayer of modifier be attached to the surface of the particle to make it compatible with the organic matrix and avoid particle agglomeration. The attachment procedure or reaction conditions required also depend on the surface modifier used. When employing silanes, surface treatment at elevated temperatures under acidic or basic conditions for about 1-24 hours is typical. Surface treatment agents such as carboxylic acids do not usually require elevated temperatures or extended time.

### Coupling Agents

Representative embodiments of surface treatment agents suitable for the durable compositions include compounds such as, for example, isooctyl trimethoxy-silane, N-(3-triethoxysilylpropyl)methoxyethoxyethoxyethyl carbamate (PEG3TES), Silquest A1230, N-(3-triethoxysilylpropyl)methoxyethoxyethoxyethyl carbamate (PEG2TES), 3-(methacryloyloxy)propyltrimethoxysilane, acryloyloxypropyl)trimethoxysilane, 3-(methacryloyloxy)propyltriethoxysilane, 3-(methacryloyloxy)propylmethyldimethoxysilane, 3-(acryloyloxypropyl)methyldimethoxysilane, 3-(methacryloyloxy)propyldimethylethoxysilane, 3-(methacryloyloxy)propyldimethylethoxysilane, vinyldimethylethoxysilane, phenyltrimethoxysilane, n-octyltrimethoxysilane, dodecyltrimethoxysilane, octadecyltrimethoxysilane, propyltrimethoxysilane, hexyltrimethoxysilane, vinylmethyldiacetoxysilane, vinylmethyldiethoxysilane, vinyltriacetoxysilane, vinyltriethoxysilane, vinyltriisopropoxysilane, vinyltrimethoxysilane, vinyltriphenoxysilane, vinyl-tri-t-butoxysilane, vinyltris-isobutoxysilane, vinyltriisopropenoxysilane, vinyltris(2-methoxyethoxy)silane, styrylethyltrimethoxysilane, mercaptopropyltrimethoxysilane, 3-glycidoxypropyltrimethoxysilane, acrylic acid, methacrylic acid, oleic acid, stearic acid, dodecanoic acid, 2-[2-(2-methoxyethoxy)ethoxy]acetic acid (MEEM), beta-carboxyethylacrylate, 2-(2-methoxyethoxy)acetic acid, methoxyphenyl acetic acid, and mixtures thereof.

Alternatively, surface modified nanoparticles are available commercially. For example zinc oxide treated with an organo silane is available from NanoTek® as Zinc Oxide C1 or Zinc Oxide C2. Gelest, Inc also sells functionalized metal nanoparticles such as zirconium n-butoxide, Catalog No. AKZ945, hafnium n-butoxide, Catalog No. AKH325, titanium methacrylate triisopropoxide, Catalog No. AKT877, zinc methacrylate, Catalog No. CSZN050, zirconyl dimethacrylate, Catalog No. CXZR051 and zirconium diacrylate dibutoxide, also from Gelest, Inc. Powdered nanoparticles and their colloidal dispersions are available from a variety of vendors such as NanoPhase Technologies, Nissan Chemical America and TAL Materials.

Gas-phase or wet-chemistry methods are employed such as PVS (physical vapor synthesis), NAS (NanoArc), plasma processes, flame pyrolysis, condensation processes in the gas phase, colloid techniques, precipitation processes, controlled nucleation and growth processes, sol-gel chemistry and (micro)emulsion processes for coating or functionalizing the nanoparticles.

Additionally, metal acetylacetonates and methods for their preparation are well known in the literature. See Charles R. G. et. al, J. Phys. Chem. (1958), 62, 440-444, or specifically US2004/0127690 for an economical process to make metal complexes of acetylacetone, herein incorporated entirely by reference. Acetylacetone functions as a chelant to the metal. These chelated metals are one of the embodiments envisioned as “functionalized nanoparticles”.

Furthermore, polymerizable β-diketones such as methacryloylacetylacetone can also be used to functionalize nanoparticles. The monomeric diketone may be polymerized, then complexed with the metal nanoparticle and incorporated into a high refractive index plastic composition which is formed from at least one of the high index of refraction monomers referred to above (formulae 1-6). Methods for forming the polymerizable β-diketones, polymerization and chelation may be found in Teyssie, P. et al, J. Polym. Sci. (1958), 47, p 245-251.

The surface modification of the particles in colloidal dispersion can be accomplished in a variety of ways and described in detail in U.S. Publication No. 2006/0147702 herein incorporated by reference.

The process involves the mixture of an inorganic dispersion with surface modifying agents. Optionally, a co-solvent can be added at this point, such as for example, 1-methoxy-2-propanol, ethanol, isopropanol, ethylene glycol, N,N-dimethylacetamide and 1-methyl-2-pyrrolidinone. The co-solvent can enhance the solubility of the surface modifying agents as well as the surface modified particles. The mixture comprising the inorganic sol and surface modifying agents is subsequently reacted at room or an elevated temperature, with or without mixing. In a preferred method, the mixture can be reacted at about 85° C. for about 24 hours, resulting in the surface modified solution. In a preferred method, where metal oxides are surface modified the surface treatment of the metal oxide can preferably involve the adsorption of acidic molecules to the particle surface. The surface modification of the metal oxide may take place at room temperature.

The surface modified particles can then be incorporated into the curable resin in various methods. In a preferred aspect, a solvent exchange procedure is utilized whereby the resin is added to the surface modified sol, followed by removal of the water and co-solvent (if used) via evaporation, thus leaving the particles dispersed in the polymerizable resin. The evaporation step can be accomplished for example, via distillation, rotary evaporation or oven drying.

In another aspect, the surface modified particles can be extracted into a water immiscible solvent followed by solvent exchange, if so desired.

Alternatively, another method for incorporating the surface modified nanoparticles in the polymerizable resin involves the drying of the modified particles into a powder, followed by the addition of the resin material into which the particles are dispersed. The drying step in this method can be accomplished by conventional means suitable for the system, such as, for example, oven drying or spray drying.

A combination of surface modifying agents can be useful, wherein at least one of the agents has a functional group co-polymerizable with a hardenable resin. For example, the polymerizing group can be ethylenically unsaturated or a cyclic function subject to ring opening polymerization. An ethylenically unsaturated polymerizing group can be, for example, an acrylate or methacrylate, or vinyl group.

Surface modification or functionalization may be accomplished by the techniques described in U.S. Publication Application Nos. 2006/0147674, 2006/0147703 and 2006/147702 herein entirely incorporated by reference.

In accordance with the present invention, the use of high index nanoparticles increases the refractive index of compositions incorporating the same. The combination of the functionalized or surface treated nanoparticles with high refractive index monomer may be advantageous in organic-inorganic hybrid materials as the combination may provide a better refractive index match to the inorganic component and give improved clarity and reduced haze.

### Mono(Meth)acrylate Aromatic Sulfur-Containing Monomers

For purposes of the invention, mono(meth)acrylate aromatic sulfur-containing monomers means monomers which are mono-functionalized or contain only one (meth)acrylate group. These aromatic sulfur-containing monomers serve the purpose of diluting and thus cutting the viscosity of the high refractive index compositions for lenses, films or coatings. The incorporation of sulfur in the aromatic monofunctional monomer helps to maintain the high refractive index but decrease the viscosity of the composition before polymerization. The end result is the compositions flow and spread more easily while maintaining the high refractive index character of the compositions.

Examples of mono(meth)acrylate aromatic sulfur-containing diluents are: 4-methylthiophenyl methacrylate; 3-methyl-4-methylthiophenyl methacrylate; phenyl thiomethacrylate; 4-methylthiobenzyl methacrylate; 2-(phenylthio)ethyl methacrylate, and β-(2-benzothiazolylthio)ethyl methacrylate. With the exception of 4-methylthiobenzyl methacrylate and 3-methyl-4-methylthiophenyl methacrylate, the other monofunctional sulfur-containing aromatic methacrylates are known and methods for making them are disclosed for example in J. Am. Chem. Soc. (1959), 81, 4302-4304, and J. Appl. Polym. Sci. (2000), 76, 50-54.

### Transparent High Refractive Index Plastic Compositions

The use of the new monomers of the invention are envisioned in combination with other monomers, multifunctional (meth)acrylates and crosslinking monomers. Preferably, the monomers are polyethylenic functional monomers containing two or three ethylenically unsaturated groups. For example, preferred polyethylenic functional compounds containing two or three ethylenically unsaturated groups may be generally described as the acrylic acid esters and the methacrylic acid esters of aliphatic polyhydric alcohols, such as, for example, the di- and triacrylates and the di- and trimethacrylates of ethylene glycol, triethylene glycol, tetraethylene glycol, tetramethylene glycol, glycerol, diethyleneglycol, butyleneglycol, propyleneglycol, pentanediol, hexanediol, trimethylolpropane, and tripropyleneglycol. Examples of specific suitable polyethylenic-functional monomers containing two or three ethylenically unsaturated groups include: trimethylolpropanetriacrylate (TMPTA), tetraethylene glycol diacrylate (TTEGDA), tripropylene glycol diacrylate (TRPGDA), 1,6-hexanediol dimethacrylate (HDDMA), and 1,6-hexanediol diacrylate (HDDA).

Lens forming compositions may include aromatic-containing bis(allyl carbonate) functional monomers and include bis(allyl carbonates) of dihydroxy aromatic-containing material. The dihydroxy aromatic containing material from which the monomer is derived may be one or more dihydroxy aromatic-containing compounds. The hydroxyl groups are attached directly to nuclear aromatic carbon atoms of the dihydroxy aromatic containing compounds. In particular, bisphenol A bis(allyl carbonate) is commonly used for optical lens formation. For a complete description of the type of bis(allyl carbonates) for use in optical lenses please refer to U.S. Pat. No. 6,419,873 herein incorporated entirely by reference.

The aromatic-containing bis(allyl carbonate) functional monomers may be represented by the formula:

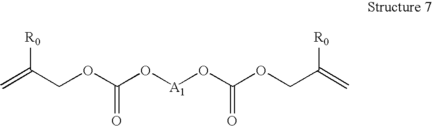

in which A₁ is the divalent radical derived from the dihydroxy aromatic-containing material and each R₀ is independently H, halo, or a C₁-C₄ alkyl group. The alkyl group is usually methyl or ethyl. Examples of R₀ include H, chloro, bromo, fluoro, methyl, ethyl, n-propyl, isopropyl and n-butyl. Most commonly R₀ is H or methyl; H is preferred. A subclass of the divalent radical A₁ which is of particular usefulness is represented by the formula:

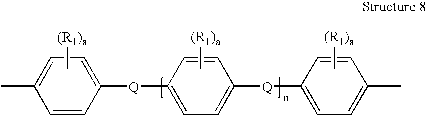

in which each R₁ is independently alkyl containing from 1 to about 4 carbon atoms, phenyl, H or halo; the average value of each (a) is independently in the range of from 0 to 4; each Q is independently oxy, sulfonyl, alkanediyl having from 2 to about 4 carbon atoms, or alkylidene having from 1 to about 4 carbon atoms; and the average value of n is in the range of from 0 to about 3. Preferably Q is methylethylidene, viz., isopropylidene.

Preferably the value of n is zero, in which case A₁ is represented by the formula:

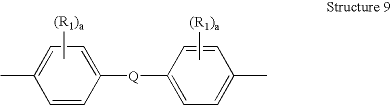

in which each R₁, each a, and Q are as discussed in respect to Structure 8. Preferably the two free bonds are both in the ortho or para positions.

The dihydroxy aromatic-containing compounds from which A₁ is derived may also be polyether-functional chain extended compounds. Examples of such compounds include alkylene oxide extended bisphenols. Typically the alkylene oxide employed is ethylene oxide, propylene oxide, or mixtures thereof. By way of exemplification, when para-bisphenols are chain extended with ethylene oxide, the bivalent radical A₁ may often be represented by the formula:

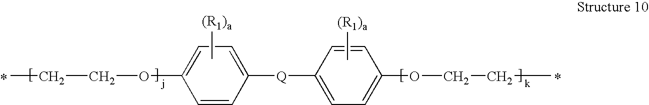

where each R₁, each a, and Q are as discussed in respect to Structure 8, and the average values of j and k are each independently in the range of from about 1 to about 4.

A preferred aromatic-containing bis(allyl carbonate) functional monomer is represented by the formula:

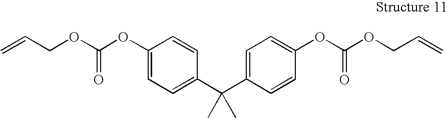

and is commonly known as bisphenol A bis(allyl carbonate).

Structure 12 may be used as a replacement of bisphenol A bis(allyl carbonate). Thus the present invention would include transparent plastic compositions which incorporate the sulfur-containing monomers of the present invention in combination with bisphenol A bis(allyl carbonate), (structure 11) or derivatives thereof and/or structure 12, wherein n is 0 to 6 and R₁ is H or methyl.

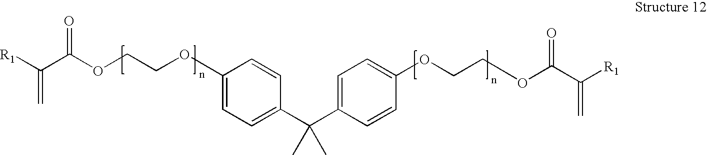

Additional high refractive index monomers may be used in addition to the sulfur-containing (meth)acrylates presently proposed. For example bromo-substituted fluorine monomers described in U.S. Application Publication No. 2006/0147703 may be included. For example acrylic acid 3-[9-(3-acryloyloxy-propyl)-2,3,7-tribromo-9H-fluoren-9-yl]-propyl ester, acrylic acid 3-{9-[3-acyloyloxy-propoxy)-propyl]-2,3,7-tribromo-9H-fluoren-9-yl}-propyl ester, 3-[9-(3-acryloyloxy-propyl)-2,7-dibromo-9H-fluoren-9-yl]-propyl ester, and 2-[9-(2-acryloyloxy-ethyl)-2,7-dibromo-9H-fluoren-9-yl]-ethyl ester may be combined with the sulfur-containing high RI monomers of the present invention.

Other high refractive index monomers which might be combined with the inventive sulfur-containing (meth)acrylates presently proposed are for example: bis(4-methacryloylthiophenyl)sulfide, bis(2-mercaptoethyl)sulfide dimethacrylate, tetrabromobisphenol A bis(2-hydroxyethyl)ether bisacrylate, 2,2′,6,6′-tetrabromobisphenol A diacrylate, pentabromophenyl acrylate, and 2-(2,4,6-tribromophenoxy)ethyl acrylate.

### Photoinitiators

Curing or crosslinking of the monomers, oligomers and optionally functionalized nanoparticles of the high refractive index composition is carried out in the presence of a photoinitiator or mixtures of photoinitiators. The photoinitiator may further include co-initiators.

Typical photoinitiators include Type I and Type II UV photoinitiators, such as the substituted acetophenone, benzoins, phosphine oxides, benzophenone/amine combinations, and other photoinitiator classes well known to those in the art. Exemplary photoinitiators include IRGACURE 819, Darocure 1173 or TPO also supplied by Ciba Specialty Chemical Corporation.

In general however, a photoinitiator for initiating the polymerization of the lens forming composition preferably exhibits an absorption spectrum over the 300-400 nm range. High absorptivity of a photoinitiator in this range, however, is not desirable, especially when casting a thick lens. The following are examples of illustrative photoinitiator compounds: methyl benzoylformate, 2-hydroxy-2-methyl-1-phenylpropan-1-one, 1-hydroxycyclohexyl phenyl ketone, 2,2-di-sec-butoxyacetophenone, 2,2-diethoxyacetophenone, 2,2-diethoxy-2-phenyl-acetophenone, 2,2-dimethoxy-2-phenyl-acetophenone, benzoin methyl ether, benzoin isobutyl ether, benzoin, benzil, benzyl disulfide, 2,4-dihydroxybenzophenone, benzylideneacetophenone, benzophenone and acetophenone. Preferred photoinitiator compounds are 1-hydroxycyclohexyl phenyl ketone (which is commercially available from Ciba Specialty Chemicals Corp. as IRGACURE 184), methyl benzoylformate (which is commercially available from Polysciences, Inc.), or mixtures thereof.

Co-initiators include reactive amine co-initiators such as monoacrylic amines, diacrylic amines, N-methyldiethanolamine, triethanolamine, ethyl 4-dimethylaminobenzoate, ethyl 2-dimethylaminobenzoate, n-butoxyethyl 4-dimethylaminobenzoate, p-dimethylamino benzaldehyde, N,N-dimethyl-p-toluidine, octyl p-(dimethylamino)benzoate.

Photoinitiators are used at 0.05 wt. % to about 10 wt. % of the total high refractive index composition or 0.1 to about 2 wt % are preferred.

When photocuring optical lens compositions, the amount of photoinitiator may vary from about 30 ppm to about 3000 ppm.

Ultraviolet-cast lenses are optical lenses or eyeglass lenses which are formed by ultraviolet (UV) curing a polymerizable liquid composition with a photoinitiator in a mold cavity. The method and typical composition for said UV-cast lenses are explained in great detail in U.S. Pat. Nos. 6,964,479 and 6,419,873 herein incorporated entirely by reference.

The polymerizable lens forming composition will also typically include aromatic-containing bis(allyl carbonate) functional monomer and at least one polyethylenic-functional monomer containing two ethylenically unsaturated groups selected from acrylate or methacrylate.

### Crosslinkers

The transparent high refractive index compositions for UV-cast lenses, films or coatings will normally contain crosslinkers. The crosslinking agents are selected from a wide variety of di- or polyfunctional moieties which are capable of crosslinking monomer species. The crosslinking agent may be an ethylenically unsaturated monomer. The ethylenically unsaturated monomer is preferably a multifunctional ethylenically unsaturated ester of (meth)acrylic acid selected from the group consisting of a difunctional ethylenically unsaturated ester of acrylic or methacrylic acid, a trifunctional ethylenically unsaturated ester of acrylic or methacrylic acid, a trifunctional ethylenically unsaturated ester of acrylic or methacrylic acid, a tetrafuntional ethylenically unsaturated ester of acrylic or methacrylic acid, and combinations thereof.

The compositions of the present invention specifically directed to UV-cast lenses are formed from at least one high refractive index monomer selected from the group consisting of

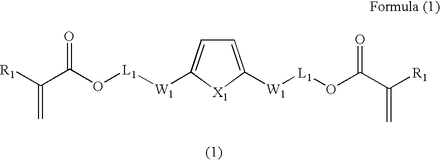

where
L₁ is defined as C₁-C₆ alkylene optionally interrupted by sulfur and/or oxygen,
W₁ is a bond, sulfur or oxygen
with the proviso that -L₁-W₁— or —W₁-L₁- contain at least one —S—, —SO₂— or —SO—,

### X₁ is S, SO or SO₂, and

R₁ is independently H or CH₃,

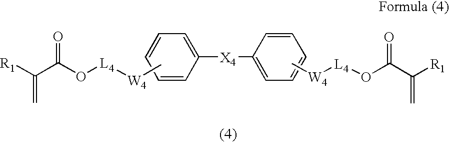

wherein
X₄ is a divalent linking group defined as —SO₂—, —SO—, —S—, —C(CH₃)₂—, —(CH₂)ₙ—S—(CH₂)ₙ—, —(CH₂)ₙ—SO—(CH₂)ₙ—, —(CH₂)ₙ—SO₂—(CH₂)ₙ—, —S—(CH₂)ₙ—S—, —SO—(CH₂)ₙ—SO— or —SO₂—(CH₂)ₙ—SO₂—,
n is 1-4,
W₄ is defined as a bond, sulfur, oxygen or a divalent linking group selected from the group consisting of —OCO—, —COO—, —CO—, —SO—, —SO₂—, —OCOO—, —OOCO—, —CONR₃—, —NR₃CO—, —SCONR₃—, —R₃NOCS—, —NR₃COS—, —SOCNR₃—, —CSO—, —OSC—, —COS—, —SOC—, —OCO—, —COO—, —CSS—, —SSC—, —SCOO—, —OOCS—, —OCONR₃— and —R₃NOCO—,
L₄ is C₁-C₁₀ alkylene which is optionally interrupted by oxygen, —S—, —SO₂—, —SO—, oxygen or W₄,
or
L₄ is a branched or linear C₁-C₄ alkylene substituted by OH, OR₄ or SR₄,
R₃ is defined independently as hydrogen or CH₃,
R₄ is branched or linear C₁-C₄ alkyl or substituted or unsubstituted phenyl, and
R₁ is independently H or CH₃.

It is preferably that at least one of -L₄-W₄— or —W₄-L₄- contains at least one sulfur.

and

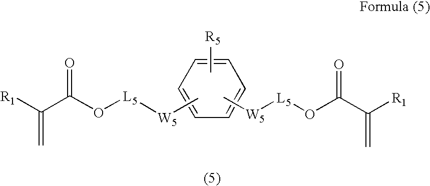

wherein
W₅ is a bond, oxygen of sulfur or a divalent linking group selected from the group consisting of —OCO—, —COO—, —CO—, —SO—, —SO₂—, —OCOO—, —OOCO—, —CONR₃—, —NR₃CO—, —SCONR₃—, —R₃NOCS—, —NR₃COS—, —SOCNR₃—, —CSO—, —OSC—, —COS—, —SOC—, —OCO—, —COO—, —CSS—, —SSC—, —SCOO—, —OOCS—, —OCONR₃— and —R₃NOCO—,
L₅ is C₁-C₁₀ alkylene optionally interrupted by oxygen, —S—, —SO₂—, —SO— or W₅,
or L₅ is a branched or linear C₁-C₄ alkylene substituted by OH, OR₄ or SR₄,
R₃ is defined independently as H or CH₃,
R₄ is branched or linear C₁-C₄ alkyl or substituted or unsubstituted phenyl,
R₅ is hydrogen or branched or linear C₁-C₄ alkyl, and
R₁ is independently H or CH₃,
with the proviso that at least one of the -L₅-W₅— or —W₅-L₅- contains at least one sulfur.

L₅ interrupted by —S—, —SO₂—, —SO—, oxygen or by a linking group may be for example —CH₂—CH₂—SO—CH₂—, —CH₂—CH₂—SO₂—CH₂—, —CH₂—CH₂—SO—CH₂—CH₂—, —CH₂—CH₂—SO—CH₂—CH₂—CH₂—CH₂—, —CH₂—CH₂—SO₂—CH₂—CH₂—, —CH₂—CH₂—O—CONH—CH₂—CH₂—, —CH₂—CH₂—NHCOO—, —CH₂CH₂—NHCOS—CH₂—, —CH₂CH₂—O—CH₂—CH₂—NHCOS—CH₂— and —CH₂CH₂—S—CH₂—CH₂—NHCOS—CH₂—CH₂.

L₅-W₅— or -L₅-W₅— may be for example, —CH₂CH₂—S—CH₂—CH₂—NHCOS—, —SOCHN—CH₂—CH₂—S—CH₂—CH₂—, —CH₂—CH₂—S—CH₂—CH₂—OCONR₃—, —CH₂—CH₂—NHCOS—CH₂—CH₂—S—, —CH₂—CH₂—O—CH₂—CH₂—S—, —CH₂—CH₂—S—, —S—CH₂—CH₂— and —CH₂CH₂—O—CH₂—CH₂—S—CH₂—.

The incorporation of high index of refraction monomers represented by formulae (1), (4), (5) or mixtures thereof into polymerizable lens forming compositions for UV-cast optical lenses is highly desirable. Firstly, incorporation of high index of refraction acrylic monomers into the compositions increases speed of cure and improves production efficiency. Secondly, a high RI composition affords thinner and lighter lenses for the same focusing power.

Also, incorporation of the sulfur-containing monomers into the cast lens, covalently bonds the sulfur within the polymer. Thus the formed polymer is substantially odor free. This is a big advantage when further milling, grinding or cutting of the lenses is required.

By high index of refraction, it is meant that the monomer has a refractive index above 1.58 and preferable above 1.60.

If W₂, W₃, W₄, W₅ is S, SO or SO₂, it is preferable that L₂, L₃, L₄ or L₅ respectively is a C₁-C₁₀ alkylene interrupted by a divalent linking group selected from the linking groups consisting of —SO—, —SO₂—, —CSO—, —OSC—, —COS—, —CSS—, —SSC—, —SCOO—, —OOCS—, —OCO—, —COO—, —OCONR₃— and —R₃NOCO—.

The most preferred divalent linking group for W₂, W₃, W₄ or W₅ is —CONR₃—, —NR₃CO—, —SO—, —SO₂—, —CSO—, —OSC—COS—, —CSS—, —SSC—, —SCOO—, —OOCS—, —SCONR₃—, —R₃NOCS—, —NR₃COS—, —COS— and —SOC—.

It is also preferable that R₃ is hydrogen.

Other additives known for their use in optical lenses, transparent coatings and films may be included in the present compositions. For example, UV sensitizers, oxygen scavengers, and other components useful in free radical curing may be employed as known in the art. Other optional additives include antioxidants, UV absorbers, surfactants, other dispersants, colorants, pigments, and other particles, other photoinitiators, and other ingredients known in the art.

The application of films or coatings may be applied using a variety of techniques, including dip coating, forward and reverse roll coating, wire wound rod coating, and die coating. Die coaters include knife coaters, slot coaters, slide coaters, fluid bearing coaters, slide curtain coaters, drop die curtain coaters, and extrusion coaters among others. Spin coating and knife coating is also envisioned.

Coatings can be applied as a single layer or as two or more superimposed layers.

The invention is further illustrated, but not thereby limited, by the Examples given below.

### EXAMPLES

### Synthesis of High Refractive Index Monomers

### Example 1

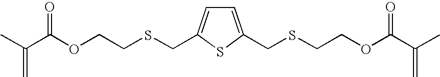

### 2,5-bis(Methacryloyloxyethylthiomethyl)thiophene

A stream of dry hydrochloric acid is bubbled vigorously through an aqueous solution of 37% formaldehyde (182 g; 2.24 moles) and concentrated HCl (147 ml) allowing the temperature to rise to 60° C. and the density to 1.18 g/cm³. The mixture is cooled to 30° C., whereupon thiophene (150 g; 1.79 moles) is added slowly with stirring and cooling to maintain the temperature between 25° C. and 30° C. After thiophene addition is complete, the mixture is stirred for an additional 20 min, the lower oily layer is separated, washed with cold water and distilled on a Vigreux column. The first fraction (46.4 g) is distilled at 30° C. and 1.2 mbar as a clear, colorless liquid, identified by GC and ¹H NMR as pure 2-chloromethylthiophene; ¹H NMR (CDCl₃, δ ppm) 7.33 (d, 1H), 7.10 (d, 1H), 6.98 (dd, 1H), 4.83 (s, 2H). The second fraction (120.4 g; yield 60%) is distilled at 80° C. and 1.2 mbar as a clear, colorless liquid which solidifies upon standing, mp 36-37° C., and is identified by GC and ¹H NMR as the desired 2,5-bis(chloromethyl)thiophene; ¹H NMR (CDCl₃, δ ppm) 6.93 (s, 2H), 4.76 (s, 4H).

2,5-Bis(chloromethyl)thiophene (100 g; 0.55 moles) is added dropwise to an aqueous solution of 45% sodium mercaptoethanol (260 g; 1.16 moles) is placed in a round-bottomed flask fitted with overhaul stirring, addition funnel and thermocouple, under a nitrogen atmosphere. During addition the temperature is raised to 50° C. The reaction mixture is stirred for an additional 5 hours at 50° C., extracted with ether, washed with 5% aqueous NaOH and cold water, and dried over Na₂SO₄. Solvent is removed giving 2,5-bis(hydroxyethylthiomethyl)thiophene as a thick liquid (136.5 g; yield 94%; n_{D}²⁵ 1.6150). ¹H NMR (CDCl₃, δ ppm) 6.73 (s, 2H), 3.87 (s, 4H), 3.66 (t, 4H), 2.68 (t, 4H), 2.10 (s, 2H).

Methacryloyl chloride (62 g of 97% purity; 0.58 moles) is added dropwise to a solution of 2,5-bis(hydroxyethylthiomethyl)thiophene (60.7 g; 0.23 moles) and triethylamine (64.3 g; 0.64 moles) in CH₂Cl₂ (500 ml) at 0-5° C. Thereafter, the mixture is stirred at room temperature for 3 more hours. The reaction is terminated by addition of water (100 ml). The organic phase is extracted with CH₂Cl₂, washed with 5% aqueous NaOH, dried over MgSO₄ and stripped of solvent under vacuum to afford 2,5-bis(methacryloyloxyethylthiomethyl)thiophene as a pale-yellow, clear liquid (76 g; yield 83%; n_{D}²⁵ 1.5584). ¹H NMR (CDCl₃, δ ppm) 6.76 (s, 2H), 6.12 (d, 2H), 5.59 (t, 2H), 4.28 (t, 4H), 3.91 (s, 4H), 2.77 (t, 4H), 1.95 (s, 6H).

### Example 2

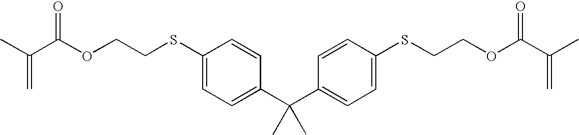

### 4,4′-Isopropylidinebis[(methacryloyloxyethylthio)benzene]

4,4′-Isopropylidinebis(thiophenol) is prepared by the Neumann-Kwart rearrangement of 4,4′-isopropylidinebis[(N,N-dimethylthiocarbamoyl)benzene] as described in J. Am. Chem. Soc. (1995), 117, 12416-12425 (24.2 g; yield 55% from bisphenol A). 4,4′-Isopropylidinebis-(thiophenol) (18.2 g; 0.07 moles) and NaOH 15% aqueous solution (40 g; 0.15 moles) are stirred for 1 h at 60° C. 2-Chloroethanol (12.1 g; 0.15 moles) is added dropwise and the reaction mixture is stirred at 60° C. for another 2 hours. The lower oily layer is separated, washed well with water and distilled at 230° C. and 0.4 mbar to give pure 4,4′-isopropylidinebis-(phenylthioethanol) (17 g; yield 70%; n_{D}²⁵ 1.6102). ¹H NMR (CDCl₃, δ ppm) 7.32 (d, 4H), 7.16 (d, 4H), 3.76 (t, 4H), 3.11 (t, 4H), 2.28 (s, 2H), 1.67 (s, 6H).

Methacryloyl chloride (10 g of 97% purity; 93 mmoles) is added dropwise to a solution of 4,4′-isopropylidinebis(phenylthioethanol) (13 g; 37 mmoles) and triethylamine (11 g; 109 mmoles) in CH₂Cl₂ (100 ml) at 0-5° C. Thereafter, the mixture is stirred at room temperature for 3 more hours. The reaction is terminated by addition of water (10 ml). The organic phase is extracted with CH₂Cl₂, washed with 5% aqueous NaOH, passed over a short silica gel plug and stripped of solvent to afford 4,4′-isopropylidinebis[(methacryloyloxyethylthio)benzene] as a clear, colorless liquid (14 g; yield 77%; n_{D}²⁵ 1.5722). ¹H NMR (CDCl₃, δ ppm) 7.31 (d, 4H), 7.15 (d, 4H), 6.07 (t, 2H), 5.56 (t, 2H), 4.32 (t, 4H), 3.17 (t, 4H), 1.92 (s, 6H), 1.65 (s, 6H).

### Example 3

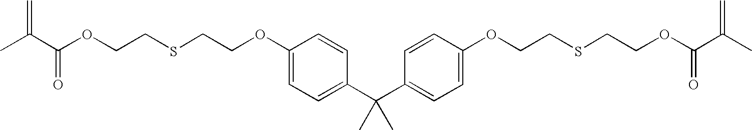

### 4,4′-Isopropylidinebis[(methacryloyloxyethylthioethyloxy)benzene]

4,4′-Isopropylidinebis(bromoethyloxybenzene) is prepared as described in J. Am. Chem. Soc. (1988), 110, 6204-6210. A solution of 4,4′-isopropylidinebis(bromoethyloxybenzene) (100 g; 0.23 moles), 2-mercaptoethanol (36 g; 0.46 moles) and triethylamine (46.6 g; 0.46 moles) in acetonitrile is stirred for 24 h at room temperature. The solvent is removed under vacuum. The crude oil is dissolved in CH₂Cl₂, washed with aqueous 5% NaOH solution, dried over anhydrous Na₂SO₄, and stripped of solvent to give 4,4′-isopropylidinebis(hydroxyethylthioethyloxybenzene) as a pale-yellow, viscous liquid (98.7 g; yield 98%). ¹H NMR (CDCl₃, δ ppm) 7.14 (d, 4H), 6.80 (d, 4H), 4.14 (t, 4H), 3.79 (q, 4H), 2.92 (t, 4H), 2.84 (t, 4H), 2.4 (s, 2H), 1.64 (s, 6H). Methacryloyl chloride (10 g of 97% purity; 93 mmoles) is added dropwise to a solution of 4,4′-isopropylidinebis(hydroxyethylthioethyloxybenzene) (19.6 g; 45 mmoles) and triethylamine (11 g; 109 mmoles) in CH₂Cl₂ (400 ml) at 0-5° C. The mixture is stirred at room temperature for 3 more hours. After addition of water (200 ml), the crude is extracted with CH₂Cl₂, washed with aqueous 5% NaOH solution, passed over a short silica gel plug and stripped of solvent to give 4,4′-isopropylidinebis[(methacryloyloxyethylthioethyloxy)benzene] as a clear liquid (20.6 g; yield 80%; n_{D}²⁵ 1.566). ¹H NMR (CDCl₃, δ ppm) 7.13 (d, 4H), 6.80 (d, 4H), 6.14 (d, 2H), 5.60 (t, 2H), 4.35 (t, 4H), 4.14 (t, 4H), 2.94 (t, 4H), 2.88 (t, 4H), 1.98 (s, 6H), 1.62 (s, 6H).

### Example 4

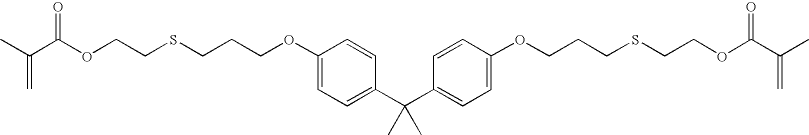

### 4,4′-Isopropylidinebis(methacryloyloxyethylthiopropyloxybenzene)

Bisphenol A (115 g; 0.5 moles), 1,3-dibromopropane (303 g; 1.5 moles) and K₂CO₃ (414 g; 1.5 moles) in acetone (500 ml) are stirred under reflux until TLC shows complete conversion of the bisphenol A (R_{f} 0.88, eluent CH₂Cl₂). The crude is stripped of solvent, dissolved in CH₂Cl₂, filtered of KBr, washed with water and dried. Evaporation of solvent gives 4,4′-isopropylidinebis(bromopropyloxybenzene) as a light-yellow liquid (205 g; yield 87%). ¹H NMR (CDCl₃, δ ppm) 7.16 (d, 4H), 6.84 (d, 4H), 4.10 (t, 4H), 3.62 (t, 4H), 2.22 (t, 4H), 1.67 (s, 6H).

A solution of 2-mercaptoethanol (44.5 g; 0.57 moles) and aqueous NaOH (22.8 g in 100 ml water; 0.57 moles) is warmed up to 60° C., stirred for 1 hour, mixed with an ethanolic solution of 4,4′-isopropylidinebis(bromopropyloxybenzene) (127 g; 0.27 moles) and then stirred for another 5 hours at 65° C. The reaction mixture is cooled to room temperature, mixed with water (200 ml), extracted with CH₂Cl₂, dried over MgSO₄ and stripped of solvent to give 4,4′-isopropylidinebis(hydroxyethylthiopropyloxybenzene) as a viscous, slight-yellow to colorless liquid (87 g; yield 90%). ¹H NMR (CDCl₃, δ ppm) 7.14 (d, 4H), 6.80 (d, 4H), 4.04 (t, 4H), 3.74 (t, 4H), 2.72 (q, 6H), 2.08 (m, 6H), 1.62 (s, 6H).

A mixture of 4,4′-isopropylidinebis(hydroxyethylthiopropyloxybenzene) (104.6 g; 0.225 moles), methacrylic acid (45 g; 0.55 moles), and p-toluenesulfonic acid (10 g) in toluene (300 ml) are refluxed until the calculated amount of water is taken out of the reaction. The reaction crude is diluted with CH₂Cl₂, washed with 5% aqueous NaOH, dried, filtered and stripped of solvent under vacuum to give 4,4′-isopropylidinebis[(methacryloyloxyethylthioproyloxy)benzene] as a clear, slight-yellow liquid (120 g; yield 87%; n_{D}²⁵ 1.562). ¹H NMR (CDCl₃, δ ppm) 7.11 (d, 4H), 6.80 (d, 4H), 6.14 (d, 2H), 5.60 (t, 2H), 4.31 (t, 4H), 4.00 (t, 4H), 2.79 (t, 8H), 2.08 (t, 4H), 1.95 (s, 6H), 1.62 (s, 6H).

### Example 5

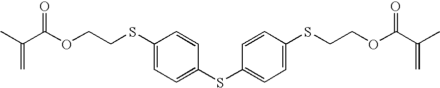

### 4,4′-Bis(methacryloyloxyethylthio)diphenylsulfide

4,4′-(Thiophenyl)sulfide (100 g; 0.4 moles; available from Sumitomo Seika) is added to a solution of NaOH (32 g; moles) in water (200 ml). The mixture is warmed up to 60° C. and stirred for an additional 1 h until a clear solution. 2-Chloroethanol (70 g; 0.87 moles) is added dropwise for 1.5 h alongside with more water (100 ml). After the addition is complete, the reaction mixture is stirred at 60° C. for another 1.5 hours, cooled to room temperature and filtered to give 4,4′-bis(hydroxyethylthiophenyl)sulfide as pure, white crystals (121 g; yield 90%). ¹H NMR (CDCl₃, 6 ppm) 7.32 (d, 4H), 7.25 (d, 4H), 3.78 (t, 4H), 3.13 (t, 4H), 1.78 (s, broad, 2H).

4,4′-Bis(hydroxyethylthiophenyl)sulfide (100 g; 0.3 moles), methyl methacrylate (100 g; 1 mole), and 2,4-dimethyl-6-tert-butyl-phenol (0.1 g) are charged in a reactor and dried by azeotropically distilling the water with cyclohexane until the water content of the reaction mixture is less then 500 ppm. Titanium isopropoxide (2 g) is added and the reaction is advanced by heating at 90-92° C. and continuously removing the methanol/cyclohexane azeotrope using a rectifying column until the desired conversion is achieved. Throughout the drying process and transesterification reaction, a steady stream of air is supplied to the reaction vessel as an additional polymerization inhibitor. The reaction mixture is then vacuum distilled to remove excess methyl methacrylate and cyclohexane, and mixed vigorously for 2 h with 20 ml water at 50° C. The resulting white titanium oxide precipitate is filtered to leave behind the desired product as a clear, light-yellow liquid (120 g; yield 90%; n_{D}²⁵ 1.6097). ¹H NMR (CDCl₃, δ ppm) 7.32 (d, 4H), 7.23 (d, 4H), 6.06 (d, 2H), 5.56 (d, 4H), 4.31 (t, 4H), 3.12 (t, 4H), 1.91 (s, 6H).

### Example 6

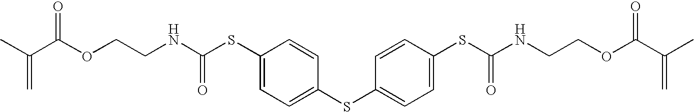

### 4,4′-Bis(methacryloyloxyethylcarbamoylthio)diphenylsulfide

A mixture of 4,4′-(thiophenyl)sulfide (12.5 g; 0.05 moles), 2-isocyanato methacrylate (34.1 g; 0.22 moles), and triethylamine (0.9 g) in toluene (150 ml) is stirred at room temperature with instantaneous formation of a voluminous white solid product. The reaction mixture is filtered and the mother liquors are placed in the refrigerator when additional solid crystallized. The combined solids afforded the desired product as a pure, white solid (25.2 g; yield 90%). ¹H NMR (CDCl₃, 6 ppm) 7.49 (d, 4H), 7.36 (d, 4H), 6.09 (d, 2H), 5.70 (t, 2H), 5.62 (d, 4H), 4.25 (t, 4H), 3.60 (q, 4H), 1.94 (s, 6H).

### Example 7

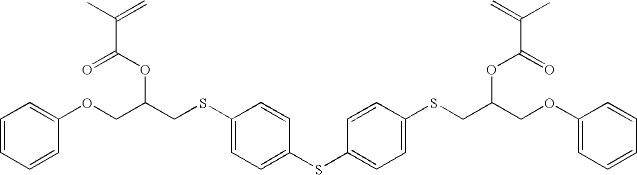

### 4,4′-Bis[2-(phenyloxymethyl)-2-(methacryloyloxy)ethylthio]diphenylsulfide

4,4′-(Thiophenyl)sulfide (12.6 g; 0.05 moles) is added to phenyl glycidyl ether (15 g; 0.1 moles) and heated to 110° C. when it became a clear liquid. The mixture is kept at 110° C. for about 6 h during which time several drops of BF₃× etherate 48% are added every one hour to catalyze the epoxide ring opening. Upon completion of reaction, the crude is cooled to room temperature to give 4,4′-bis[2-(phenyloxymethyl)-2-(hydroxy)ethylthio]diphenylsulfide as a grey-white solid (25 g; yield 90%). ¹H NMR (CDCl₃, δ ppm) 7.25 (m, 12H), 6.96 (t, 2H), 6.85 (d, 4H), 4.10 (m, 8H), 3.20 (ddd, 2H), 1.35 (s, broad, 2H).

Methacryloyl chloride (11 g of 97% purity; 107 mmoles) is added dropwise to a solution of 4,4′-bis[2-(phenyloxymethyl)-2-(hydroxy)ethylthio]diphenylsulfide (27.6 g; 50 mmoles) and triethylamine (13.5 g; 134 mmoles) in CH₂Cl₂ (100 ml) at 0-5° C. The mixture is stirred at room temperature for 3 h. After addition of water (50 ml), the crude is extracted with CH₂Cl₂, washed with aqueous 5% NaOH solution, passed over a short silica gel plug and stripped of solvent to give 4,4′-bis[2-(phenyloxymethyl)-2-(methacryloyloxy)ethylthio]diphenylsulfide as a clear, slight-yellow liquid (29.3 g; yield 85%; n_{D}²⁵ 1.621). ¹H NMR (CDCl₃, δ ppm) 7.20 (m, 12H), 6.90 (t, 2H), 6.80 (d, 4H), 6.00 (s, 2H), 5.48 (s, 2H), 5.22 (quintet, 2H), 4.21 (t, 2H), 3.39 (dd, 2H), 1.83 (s, 6H), 1.51 (s, 4H).

### Example 8

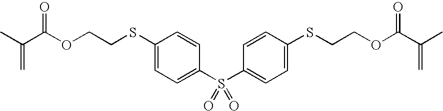

### 4,4′-Bis(methacryloyloxyethylthio)diphenylsulfone

4,4′-Bis(chlorophenyl)sulfone (263 g; 0.92 moles), 2-mercaptoethanol (165 g; 2.12 moles), K₂CO₃ (313 g; 2.27 moles) and dimethylacetamide (791 g) are charged into a 2 liter round-bottom flask equipped with mechanical stirrer, thermocouple and condenser. The reaction mixture is stirred at 15° C. for 5 h, cooled down to room temperature and poured into water (2 L) when the desired product precipitates. The solid cake is filtered and washed well with water to give after drying 4,4′-bis(hydroxyethylthio)diphenylsulfone as pure, white crystals (333.6 g; yield 98%). ¹H NMR (CDCl₃, δ ppm) 7.80 (d, 4H), 7.40 (d, 4H), 3.82 (t, 4H), 3.21 (t, 4H), 1.76 (s, 2H).

4,4′-Bis(hydroxyethylthio)diphenylsulfone (112 g; 0.3 moles), methyl methacrylate (100 g), 2,4-dimethyl-6-tert-butyl-phenol (0.1 g), cyclohexane (80 ml) and dibutyltin oxide (2.15 g) are charged in a reactor. The reaction is advanced by heating at 90-92° C. and continuously removing the methanol/cyclohexane azeotrope using a rectifying column until the desired conversion is achieved. Throughout the drying process and transesterification reaction, a steady stream of air is supplied to the reaction vessel as an additional polymerization inhibitor. The reaction is followed up by TLC. When conversion is complete, the crude is cooled to room temperature, mixed with methanol and placed in the refrigerator overnight where 4,4′-bis(methacryloyloxyethyl)diphenylsulfone precipitates as pure, white crystals (144.2 g; yield 95%; n_{D}²⁵ 1.6097; mp 45° C.). ¹H NMR (CDCl₃, δ ppm) 7.81 (d, 4H), 7.42 (d, 4H), 6.04 (d, 2H), 5.38 (t, 2H), 4.35 (t, 4H), 3.28 (t, 4H), 1.90 (s, 6H).

### Example 9

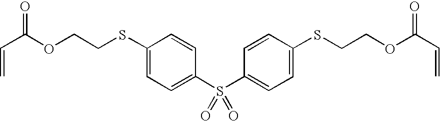

### 4,4′-Bis(acryloyloxyethylthio)diphenylsulfone

Distilled acryloyl chloride (6.3 g; 70 mmoles) is added dropwise to a vigorously stirred suspension of 4,4′-bis(hydroxyethylthio)diphenylsulfone (10 g; 27 mmoles) and tetrabutylammonium bromide (2.3 g; 7 mmoles) in 50% aqueous KOH (5.6 g; 100 mmoles) and dichloromethane (50 g), cooled at 4° C. After completion of addition the mixture is stirred for an additional 2 hours at 4-8° C., and then overnight at room temperature with an air sparge. The reaction crude is decanted, washed several times with water, dried over MgSO₄ and stripped of solvent under vacuum and a continuous air sparge, to give the product as a clear, colorless, very viscous oil (12.6 g; yield 97%). ¹H NMR (500 MHz, CDCl₃, δ ppm) 7.81 (d, 4H), 7.41 (d, 4H), 6.36 (d, 2H), 6.05 (k, 2H), 6.82 (d, 2H) 4.34 (t, 4H), 3.25 (t, 4H).

### Example 10

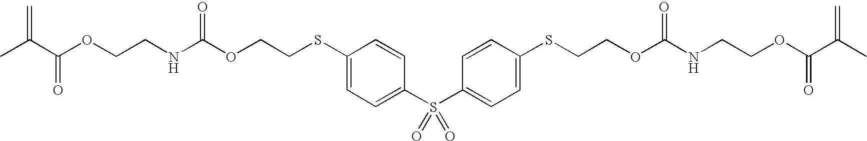

### 4,4′-Bis(methacryloyloxyethylcarbamatoethylthio)phenylsulfone

A mixture of 4,4′-bis(hydroxyethylthio)diphenylsulfone (50 g; 0.14 moles), 2-isocyanatoethyl methacrylate (43.4 g; 0.28 moles) and triethylamine (0.3 g, 3 mmols) in acetonitrile is stirred at room temperature until a clear solution is obtained. The crude is passed on a short silica gel plug and the solvent is removed at the rotary evaporator to give the desired product as a viscous, clear, colorless liquid which solidifies upon standing (93.3 g; yield 98%; mp 75° C.; n_{D}²⁵ 1.580). ¹H NMR (CDCl₃, δ ppm) 7.79 (d, 4H), 7.40 (d, 4H), 6.11 (d, 2H), 5.59 (t, 2H), 5.02 (m, 2H), 4.23 (m, 8H), 3.48 (m, 4H), 3.20 (t, 4H), 1.96 (s, 6H).

### Example 11

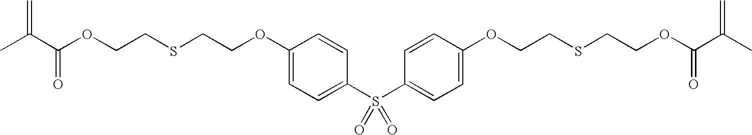

### 4,4′-Bis(methacryloyloxyethylthioethyloxy)diphenylsulfone

PCl₅ (104 g; 0.5 moles) is added in small portions to a solution of 4,4′-bis(hydroxyethyloxy)-diphenylsulfone (86.1 g; 0.25 moles) in CCl₄ (400 ml). After the addition is complete, the mixture is stirred overnight at 45-60° C., cooled to room temperature, and poured under vigorous stirring in cold water. The white solid precipitated is filtered, recrystallized from DMF and dried to give pure 4,4′-bis(chloroethyloxy)diphenylsulfone (86 g; yield 90%). ¹H NMR (CDCl₃, δ ppm) 7.85 (d, 4H), 6.96 (d, 4H), 4.25 (t, 4H), 3.81 (t, 4H).

A mixture of 4,4′-bis(chloroethyloxy)diphenylsulfone (80 g; 0.21 moles), 2-mercaptoethanol (35.2 g; 0.45 moles) and K₂CO₃ (62.2 g; 0.45 moles) in DMA (300 g) is stirred at 150° C. under N₂ for 2 hours and then at room temperature for another 4 hours. The reaction crude is into water and the white solid precipitated is filtered and dried under vacuum to give 4,4′-bis(hydroxyethylthioethyloxy)diphenylsulfone (90.4 g; yield 94%). ¹H NMR (CDCl₃, δ ppm) 7.83 (d, 4H), 6.94 (d, 4H), 4.18 (t, 4H), 3.78 (t, 4H), 2.91 (t, 4H), 2.82 (t, 4H), 1.94 (s, 2H).

Methacryloyl chloride (27 g of 97% purity; 0.25 moles) is added dropwise to a solution of 4,4′-isopropylidinebis(hydroxyethylthioethyloxybenzene) (46 g; 0.1 moles) and triethylamine (25.3 g; 0.25 moles) in CH₂Cl₂ (400 ml) at 0° C. The mixture is stirred at room temperature for 3 more hours. After addition of water (500 ml), the crude is extracted with CH₂Cl₂, washed with aqueous 5% NaOH solution, passed over a short silica gel plug and stripped of solvent to give 4,4′-bis(methacryloyloxyethylthioethyloxy)diphenylsulfone as a clear, slight-yellow to colorless viscous liquid (53 g; yield 90%). ¹H NMR (CDCl₃, δ ppm) 7.84 (d, 4H), 6.95 (d, 4H), 6.10 (s, 2H), 5.57 (s, 2H), 4.33 (t, 4H), 4.17 (t, 4H), 2.96 (t, 4H), 2.88 (t, 4H), 1.93 (s, 6H).

### Example 12

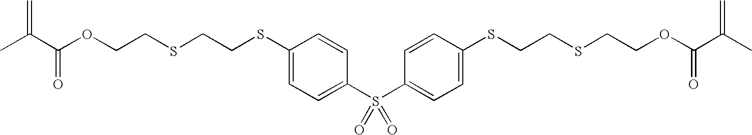

### 4,4′-Bis(methacryloyloxyethylthioethylthio)diphenylsulfone

PCl₅ (31.2 g; 150 mmoles) is added in small portions to a solution of 4,4′-bis(hydroxyethylthio)diphenylsulfone (25 g; 68 mmoles) in CCl₄ (150 ml). After the addition is complete, the mixture is stirred overnight at 45-60° C., cooled to room temperature, and poured under vigorous stirring in cold water. The white solid precipitated is filtered, washed with methanol and dried to give pure 4,4′-bis(chloroethylthio)diphenylsulfone (25 g; yield 90%). ¹H NMR (CDCl₃, δ ppm) 7.82 (d, 4H), 7.40 (d, 4H), 3.66 (t, 4H), 3.33 (t, 4H).

A mixture of 4,4′-bis(chloroethylthio)diphenylsulfone (10 g; 25 mmoles), 2-mercaptoethanol (7 g; 90 mmoles) and K₂CO₃ (12.3 g; 90 mmoles) in DMA (50 g) is stirred at 150° C. under N₂ for 2.5 hours. The reaction crude is cooled to room temperature, mixed with water, extracted with CH₂Cl₂, washed with 5% aqueous NaOH, dried, filtered and stripped of solvent to give 4,4′-bis(hydroxyethylthioethylthio)diphenylsulfone as a viscous, light-yellow to colorless liquid (11.5 g; 94%). ¹H NMR (CDCl₃, 8 ppm) 7.80 (d, 4H), 7.37 (d, 4H), 3.72 (t, 4H), 3.22 (t, 4H), 2.78 (m, 8H), 2.34 (s, 2H).

Methacryloyl chloride (4.2 g of 97% purity; 39 mmoles) is added dropwise to a solution of 4,4′-isopropylidinebis(hydroxyethylthioethylthiobenzene) (8.91 g; 18 mmoles) and triethylamine (4.05 g; 40 mmoles) in CH₂Cl₂ (40 ml) at 0-5° C. The mixture is stirred at room temperature for 3 more hours. After addition of water (50 ml), the crude is extracted with CH₂Cl₂, washed with aqueous 5% NaOH solution, passed over a short silica gel plug and stripped of solvent to give 4,4′-bis(methacryloyloxyethylthioethylthio)diphenylsulfone as a clear, slight-yellow to colorless viscous liquid (12.4 g; yield 80%). ¹H NMR (CDCl₃, 8 ppm) 7.82 (d, 4H), 7.35 (d, 4H), 6.13 (s, 2H), 5.59 (s, 2H), 4.31 (t, 4H), 3.20 (m, 4H), 2.84 (m, 8H), 1.94 (s, 6H).

### Example 13

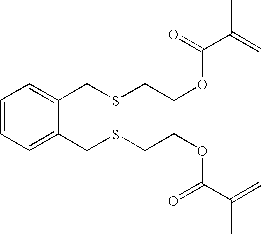

### 1,2-Bis(methacryloyloxyethylthiomethyl)benzene

1,2-Bis(bromomethyl)benzene (84 g; 0.32 moles) is added gradually to the Na salt of 2-mercaptoethanol (150 g of 45.5% aqueous solution as MERCASOL L from Chevron-Phillips; 0.68 moles) at 60° C. The reaction crude is stirred for 4 hours at 60° C., cooled to room temperature, and extracted with ethyl acetate. The organic phase is washed with water, dried over MgSO₄, filtered and vacuum distilled to give 1,2-bis(hydroxyethylthiomethyl)benzene as a pale yellow liquid (63 g; yield 76%; bp 178° C. at 1.2 mbar). ¹H NMR (CDCl₃, 8 ppm) 7.24 (m, 4H), 3.92 (s, 4H), 3.72 (t, 4H), 2.71 (t, 4H), 2.62 (s, 2H).

1,2-Bis(hydroxyethylthiomethyl)benzene (57 g; 0.22 moles), methacrylic anhydride (82 g; 0.53 moles), triethylamine (51 g; 0.5 moles) and CH₂Cl₂ (250 ml) are mixed at room temperature for 48 h. An aqueous solution of NaHCO₃ (51 g in 627 g water) is mixed in with the reaction crude and stirred for another 30 minutes. The bottom organic layer is separated, washed with water and diluted aqueous NaOH, and stripped of solvent under vacuum to give 1,2-bis(methacryloyloxyethylthiomethyl)benzene as a pale-yellow, clear liquid (85 g; yield 98%; n_{D}²⁵ 1.566). ¹H NMR (CDCl₃, 8 ppm) 7.25 (m, 4H), 6.12 (s, 2H), 5.60 (s, 2H), 4.29 (t, 4H), 3.96 (s, 4H), 2.75 (t, 4H), 1.96 (s, 6H).

### Example 14

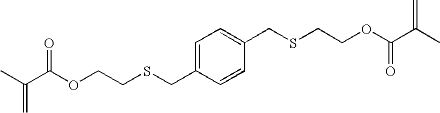

### 1,4-Bis(methacryloyloxyethylthiomethyl)benzene

1,4-Bis(bromomethyl)benzene (84 g; 0.32 moles) is added gradually to the Na salt of 2-mercaptoethanol (150 g of 45.5% aqueous solution as MERCASOL L from Chevron-Phillips; 0.68 moles) at 60° C. The reaction crude is stirred for 4 h at 60° C., cooled to room temperature, and extracted with ethyl acetate. The organic phase is washed with water, dried over MgSO₄, filtered, and recrystallized from ethanol to give 1,4-bis(hydroxyethylthiomethyl)benzene as pure, white crystals (76 g; mp 91° C.; yield 92%). ¹H NMR (CDCl₃, δ ppm) 7.29 (s, 4H), 3.72 (s, 4H), 3.66 (t, 4H), 2.65 (t, 4H), 2.03 (s, 2H).

1,4-Bis(hydroxyethylthiomethyl)benzene (57 g; 0.22 moles), methacrylic anhydride (82 g; 0.53 moles), triethylamine (51 g; 0.5 moles) and CH₂Cl₂ (250 ml) are mixed at room temperature for 48 h. An aqueous solution of NaHCO₃ (51 g in 627 g water) is mixed in with the reaction crude and stirred for another 30 minutes. The bottom organic layer is separated, washed with water and with diluted aqueous NaOH, and striped of solvent under vacuum to give 1,4-bis(methacryloyloxyethylthiomethyl)benzene as a pale-yellow, clear liquid (85 g; yield 98%; n_{D}²⁵ 1.565). ¹H NMR (CDCl₃, δ ppm) 7.29 (s, 4H), 6.12 (s, 2H), 5.58 (s, 2H), 4.28 (t, 4H), 3.77 (s, 4H), 2.71 (t, 4H), 1.96 (s, 6H).

### Example 15

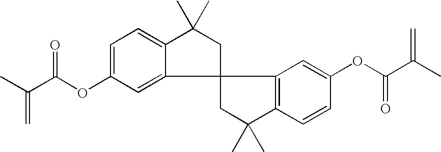

### 1,1,1′,1′-Tetramethyl-5,5′-dihydroxy-3,3′-spirobiindane dimethacrylate

Bisphenol A (50 g; 0.22 moles) and CH₃OSO₂H (3 g) are heated at 135° C. for 3 hours. The reaction mixture is poured into water with stirring and filtered. The solid is recrystallized from dichloromethane to give 1,1,1′,1′-tetramethyl-5,5′-dihydroxy-3,3′-spirobiindane as pure, white crystals (11.3 g; yield 17%). ¹H NMR (CDCl₃, δ ppm) 7.03 (d, 2H), 6.70 (dd, 2H), 6.20 (d, 2H), 4.45 2.28 (dd, 4H), 1.59 (s, 2H), 1.37 (s, 6H), 1.32 (s, 6H).

Methacryloyl chloride (4.2 g of 97% purity; 39 mmoles) is added dropwise to a solution of 1,1,1′,1′-tetramethyl-5,5′-dihydroxy-3,3′-spirobiindane (4 g; 13 mmoles) and triethylamine (4 g; 40 mmoles) in CH₂Cl₂ (30 ml) at 0-5° C. The mixture is stirred at room temperature for 3 more hours. The solvent is stripped under vacuum and the residue is recrystallized from acetone-pentane to give 1,1,1′,1′-tetramethyl-5,5′-dihydroxy-3,3′-spirobiindane dimethacrylate as pure, white crystals (4.5 g; yield 78%). ¹H NMR (CDCl₃, δ ppm) 7.18 (dd, 2H), 6.96 (dd, 2H), 6.57 (dd, 2H), 6.28 (s, 2H), 5.69 (t, 2H), 2.34 (dd, 4H), 2.02 s, 6H), 1.40 (s, 6H), 1.36 (s, 6H).

### Example 16

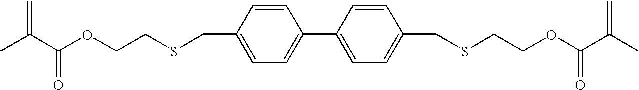

### 4,4′-Bis(methacryloyloxyethylthiomethyl)biphenyl

A mixture of 4,4′-bis(chloromethyl)biphenyl (12.5 g; 50 mmoles), 2-mercaptoethanol (8.6 g; 110 mmoles) and NaOH 45% (20 g) in DMA (20 ml) is stirred at 130° C. under N₂ for 1 hour. The reaction crude is cooled to room temperature, mixed with water and filtered to give 4,4′-bis(hydroxyethylthiomethyl)biphenyl as a white solid (16 g; 95%; mp 152° C.). ¹H NMR (DMSO-d₆, δ ppm) 7.61 (d, 4H), 7.39 (d, 4H), 4.77 (t, 2H), 3.79 (s, 4H), 3.53 (q, 4H), 2.50 (t, 4H). Methacryloyl chloride (2.95 g of 97% purity; 27.9 mmoles) is added dropwise to a vigorously stirred suspension of 4,4′-bis(hydroxyethylthiomethyl)biphenyl (3.33 g; 9.96 mmol) and tetrabutylammonium bromide (0.5 g; 1.5 mmol) in 44% aqueous KOH (1.56 g; 27.9 mmol) and dichloromethane (20 ml), cooled at 4° C. After completion of addition the mixture is stirred for an additional hour at 4-8° C., and for 2 more hours at room temperature. The reaction crude is washed with water, filtered and stripped of solvent under vacuum to give 3.3 g of crude product. Pure 4,4′-bis(methacryloyloxyethylthiomethyl)biphenyl is obtained after column chromatography (silica gel; dichloromethane) followed by preparative TLC (dichloromethane:cyclohexane 7:1; silica gel). ¹H NMR (CDCl₃, δ ppm) 7.54 (d, 4H), 7.39 (d, 4H), 6.12 (s, 2H), 5.59 (s, 2H), 4.3 (t, 4H), 3.81 (s, 4H), 2.73 (t, 4H), 1.95 (s, 6H).

### Example 17

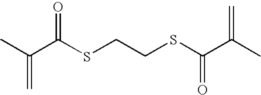

### Ethylene glycol dithiomethacrylate

A solution of 1,2-dimercaptoethane (25 g; 0.27 moles) in 15% aqueous KOH (350 g; 0.94 mole) and methacryloyl chloride (67 g; 0.62 moles) are added simultaneously to tetrabutylammonium bromide (5 g) and p-methoxyphenol (0.3 g) in CH₂Cl₂ (400 ml) cooled at 0° C. After completion of addition, the reaction mixture is stirred for another 1 hour. The organic layer is separated, mixed with cyclohexane (200 ml) and the CH₂Cl₂, is distilled under vacuum on a rotary evaporator. The crude is washed with diluted NaHCO₃, and distilled to give phenyl thiomethacrylate as a clear, colorless liquid (36 g; yield 58%; n_{D}²⁵ 1.547; bp 91° C. @ 0.5 mbar). ¹H NMR (CDCl₃, δ ppm) 6.09 (s, 2H), 5.62 (s, 2H), 3.14 (s, 4H), 1.99 (s, 6H).

### Preparation of Monofunctional Sulfur-Containing (Meth)Acrylates

### Example 18

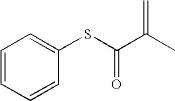

### Phenyl thiomethacrylate

A solution of thiophenol (33 g; 0.3 moles) in 10% aqueous KOH (224 g; 0.4 moles KOH) and methacryloyl chloride (38 g; 0.36 moles) are added simultaneously to tetrabutylammonium bromide (4 g) and p-methoxyphenol (0.3 g) in CH₂Cl₂ (200 ml) cooled at 0° C. After completion of addition, the reaction mixture is stirred for another 1 h. The organic layer is separated and mixed with cyclohexane (100 ml) and the CH₂Cl₂ is distilled under vacuum on a rotary evaporator. The crude is washed with diluted NaHCO₃, and distilled to give phenyl thiomethacrylate as a clear, colorless liquid (35 g; yield 66%; n_{D}²⁵ 1.5741; bp 106° C. @ 4 mm Hg). ¹H NMR (CDCl₃, δ ppm) 7.45 (s, 5H), 6.24 (s, 1H), 5.72 (s, 1H), 2.03 (s, 3H).

### Example 19

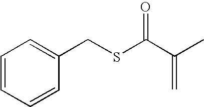

### Benzyl thiomethacrylate

Methacryloyl chloride (61 g; 0.58 moles) is added dropwise to a mixture of benzyl mercaptan (55.6 g; 0.45 moles), CH₂Cl₂ (200 ml) and 7.6% aqueous NaOH (400 g; 0.76 moles) keeping the temperature below 10° C. by cooling with ice. After addition is complete, the reaction mixture is stirred for an additional 2 hours. The organic layer is separated, washed with water, dried with anhydrous MgSO₄, and vacuum distilled to give benzyl thiomethacrylate as a clear, colorless liquid which solidifies upon storage in the refrigerator (80 g; yield 72%; n_{D}²⁵ 1.568; bp 119° C. @ 4 mm Hg). ¹H NMR (CDCl₃, δ ppm). 1.625 7.30 (m, 5H), 6.11 (d, 1H), 5.61 (d, 1H), 4.20 (s, 2H), 2.02 (s, 3H).

### Example 20

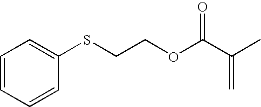

### 2-Phenylthioethyl methacrylate

2-Phenylthioethanol (154 g; 1 mole; available from Chevron-Phillips), methyl methacrylate (125 g; 1.25 mole), cyclohexane (60 g), activated carbon (2 g) and 2,4-dimethyl-6-tert-butyl-phenol (0.1 g) are charged in a reactor and dried by azeotropically by distilling the water with cyclohexane until the water content of the reaction mixture is less then 500 ppm. Titanium iso propoxide (3 g) is added and the reaction advanced by heating at 90-92° C. and continuously removing the methanol/cyclohexane azeotrope using a rectifying column until the desired conversion is achieved. Throughout the drying process and transesterification reaction, a steady stream of air is supplied to the reaction vessel as an additional polymerization inhibitor. The conversion of the reaction is followed by GC. When complete, the crude was vacuum distilled to afford 2-phenylthioethyl methacrylate as a clear, colorless liquid (200 g; yield 90%; n_{D}²⁵ 1.556; bp 110° C. @ 1.2 mbar). ¹H NMR (CDCl₃, δ ppm) 7.41 (d, 1H), 7.31 (t, 2H), 7.20 (t, 2H), 6.08 (s, 1H), 5.57 (s, 1H), 4.33 (t, 2H), 3.20 (t, 2H), 1.93 (s, 3H).

### Example 21

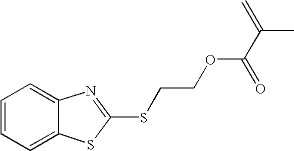

### 2-[(2-Benzothiazolyl)mercapto]ethyl methacrylate

2-Mercaptobenzothiazole (100 g; 0.56 moles), 2-chloroethyl methacrylate (85 g; 0.58 moles), NaHCO₃ (48 g; 0.57 moles), and DMF (180 g) are mixed at 90° C. for 5 h. The conversion of the reaction is followed by GC. When complete, the mixture is cooled to room temperature, mixed with 5% aqueous NaOH, filtered and extracted with diethyl ether. The top organic layer is separated, dried over MgSO₄, filtered and stripped of solvent under vacuum to afford 2-[(2-benzothiazolyl)mercapto]ethyl methacrylate as a pale-yellow liquid (140.6 g; yield 90%; n_{D}²⁵ 1.628). ¹H NMR (CDCl₃, δ ppm) 1.628 7.85 (d, 1H), 7.76 (d, 1H), 7.40 (t, 1H), 7.29 (dd, 1H), 6.12 (s, 1H), 5.38 (s, 1H), 4.55 (t, 2H), 3.679 (t, 2H), 1.93 (s, 3H).

### Example 22

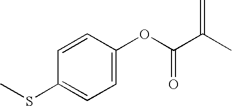

### 4-Methylthiophenyl methacrylate

A mixture of 4-(methylthio)phenol (42 g; 0.3 moles), methacrylic acid (34 g; 0.4 moles), 4-(methoxy)phenol (0.6 g) and p-toluenesulfonic acid (5 g) in xylene (80 ml) are refluxed until the calculated amount of water is taken out of the reaction. A continuous air sparge is used during reflux to prevent polymerization. The reaction crude is diluted with CH₂Cl₂, washed with 5% aqueous NaOH, dried, filtered and vacuum distilled to give 4-methylthiobenzyl methacrylate as a white, low melting solid (25 g; yield 40%; n_{D}⁴⁰ 1.561; mp 41° C.; bp 150° C. @ 5 mm Hg). ¹H NMR (CDCl₃, δ ppm) 7.30 (d, 2H), 7.08 (d, 2H), 6.35 (s, 1H), 5.76 (h, 1H), 2.47 (s, 3H), 2.06 (s, 3H). ¹³C NMR (CDCl₃, 8 ppm) 165.8, 148.6, 135.7, 135.5, 127.9, 127.3, 122.1, 18.4, 16.5.

### Example 23

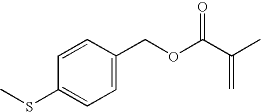

### 4-Methylthiobenzyl methacrylate

A mixture of 4-methylthiobenzyl alcohol (50 g; 0.33 moles), methacrylic acid (34 g; 0.4 moles), 4-(methoxy)phenol (0.2 g) and p-toluenesulfonic acid (0.6 g) in toluene (60 ml) are refluxed until the calculated amount of water is taken out of the reaction. A continuous air sparge is used during reflux to prevent polymerization. The reaction crude is diluted with CH₂Cl₂, washed with 5% aqueous NaOH, dried, filtered and vacuum distilled to give 4-methylthiobenzyl methacrylate as a clear, colorless liquid (40 g; yield 55%; n_{D}²⁵ 1.565; bp 106° C. @ 0.5 mbar). ¹H NMR (CDCl₃, 6 ppm) 7.32 (d, 2H), 7.25 (d, 2H), 6.15 (s, 1H), 5.59 (h, 1H), 5.16 (2, 2H), 2.49 (s, 3H), 1.98 (s, 3H). ¹³C NMR (CDCl₃, δ ppm) 167.2, 138.6, 136.1, 132.7, 128.7, 126.4, 125.8, 66.0, 18.3, 15.7.

### Example 24

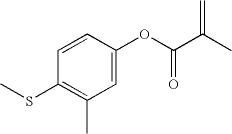

3-Methyl-4-methylthiophenyl methacrylate

A mixture of 3-methyl-4-methylthiophenol (20 g; 0.13 moles), methacrylic anhydride (24 g; 0.15 moles), and triethylamine (15.7 g) in dichloromethane (100 ml) is stirred at room temperature for one hour. The reaction crude is washed with 5% aqueous NaOH (150 ml), dried over anhydrous Na₂SO₄, filtered and vacuum distilled to give 3-methyl-4-methylthiophenyl methacrylate as a clear, colorless liquid (23 g; yield 82%; n_{D}²⁵ 1.564; bp 90° C. @ 0.56 mbar). ¹H NMR (CDCl₃, 6 ppm) 7.19 (d, 1H), 6.97 (d, 1H), 6.96 (s, 1H), 6.35 (s, 1H), 5.76 (s, 1H), 2.47 (s, 3H), 2.36 (s, 3H), 2.07 (s, 3H). ¹³C NMR (CDCl₃, 8 ppm) 165.9, 148.2, 137.4, 135.8, 134.8, 127.1, 126.1, 122.9, 119.5, 20.0, 18.4, 15.7.

### Example 25

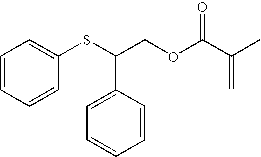

### 2-Phenyl-2-phenylthioethyl methacrylate

Styrene oxide (3.96 g; 33 mmoles) is added during one hour to a stirring solution of thiophenol (3.63 g; 33 mmoles) and gallium triflate (0.17 g; 0.33 mmoles; 1 mole %) heated at 35-40° C. The crude mixture is stirred for another 3 hours, poured into water (25 ml) and extracted with diethyl ether. The organic layer is dried over anhydrous MgSO₄, filtered, stripped of solvent and vacuum distilled to give 2-phenyl-2-phenylthioethanol as a clear oil (2 g; yield 26%; n_{D}²⁰ 1.618; bp 134-138° C. @ 0.9 mbar). ¹H NMR (CDCl₃, δ ppm) 7.31-7.20 (m, 10H), 4.29 (t, 1H), 3.89 (dd, 1H), 3.87 (dd, 1H), 1.86 (s, 1H).

2-Phenyl-2-phenylthioethanol (1.93 g; 8.4 mmoles), methyl methacrylate (12 g; 0.12 moles), dibutyltinoxide (0.05 g; 0.2 mmoles) and 4-methoxyphenol (5 mg) are stirred at 95° C. for 11 hours with an air sparge. The reaction crude is stripped of volatiles under vacuum, mixed with cyclohexane, decanted, and purified by column chromatography (silica gel; cyclohexane then cyclohexane:ethyl acetate 2:3) to afford 2-phenyl-2-phenylthioethyl methacrylate as a clear, lightly colored oil (1.17 g; yield 46%; n_{D}²⁰ 1.576). ¹H NMR (C₆D₆, δ ppm) 7.31-6.87 (m, 10H), 5.99 (s, 1H), 5.09 (q, 1H), 4.60 (dd, 1H), 4.49 (dd, 1H), 4.45 (t, 1H), 1.71 (q, 3H).

### Preparation of Hybrid UV-Curable Compositions

### Example 26

Hybrid UV-curable compositions can be made by simply blending inorganic sols with high RI acrylic monomers and organic modifiers under efficient stirring to produce a homogeneous mixture which can be used as is for coatings, or have the solvent removed under vacuum before UV-casting. Organic modifiers are monomers with ligand functionalities directly linked to the inorganic part. Examples include 2-hydroxyethyl acrylate (HEA) and methacrylate (HEMA), and 2-(acryloyloxy)ethyl acetoacetate (AAEA). The composite materials can be intended for a variety of desired properties, such as hardness, toughness, flexibility, transparency, high RI, thermal, abrasion or impact resistance.

4-Methylthiobenzyl methacrylate from Example 23 (2 g; 9 mmoles), HEMA (0.62 g; 4.8 mmoles), Zr(OisoPr)₄ (0.55 g of 70% solution in isopropanol) and Irgacure 651 (35 mg) are blended together. The homogeneous solution is cast into molds or applied on a surface as a thin film, and UV-cured to give clear, hard plastic parts.

### APPLICATION EXAMPLES

### Optical Lens

UV-curable formulations are prepared by mixing the monomers with a photoinitiator in concentration of up to 1 mole %. Suitable photoinitiators include Irgacure 819, Irgacure 651 or Irgacure 2022, available from Ciba Specialty Chemicals. The polymerizable compositions and relevant parameters and properties of the UV-cured articles are presented in Table 2.

TABLE 2
Compositions and optical and mechanical properties of UV-cured articles

| | Monomer 1 | Monomer 2 | | T_{g} | Rockwell hardness | |
|---|---|---|---|---|---|---|
| Example | [wt %] | [wt %] | n_{D} | [° C.] | [R scale] | Odor |
| 27 | Example 2 | — | 1.61 | 107 | 124 | no |
| | 100 | | | | | |
| 28 | Example 5 | — | 1.65 | 95 | 123 | no |
| | 100 | | | | | |
| 29 | Example 7 | Example 19 | 1.64 | 75 | 117 | no |
| | 75 | 25 | | | | |
| 30 | Example 8 | — | 1.63 | 130 | — | no |
| | 100 | | | | | |
| 31 | Example 13 | — | 1.60 | 85 | 120 | no |
| | 100 | | | | | |
| 32 | Example 23 | — | — | 45 | — | no |
| | 100 | | | | | |

### Refractive Index

Refractive indices are measured at 25° C. and 589 nm using an Abbe refractometer.

### Glass Transition Temperature

Glass transition temperature, T_{g}, is measured both by DSC (Differential Scanning Calorimetry) and DMA (Dynamic Mechanical Analysis).

### Differential Scanning Calorimetry

DSC is carried out on a TA Instrument DSC Q1000 calorimeter. The DSC analysis of UV-cured disks is done on circa 5-15 mg of sample in AI pans under nitrogen at atmospheric pressure, upon heating from 20° C. to 300° C. with a rate of 10° C./min.

### Dynamic Mechanical Analysis

DMA is done on a TA Instruments AR2000N rheometer. The UV-cured disks are cut into rectangles, and edges are sanded smooth to remove any small fractures. The samples are mounted in the rheometer torsion clamps, subjected to a 1 Hz oscillation, 0.5% strain, and 5 N normal force in tension, while scanning at 2° C./min from −35° C. to 125° C.

### Rockwell Hardness

Hardness is measured on the Rockwell hardness scale (ASTM D785-93). The test result is reported as a Rockwell hardness number directly related to the indentation hardness of a plastic material, with the higher the reading the harder the material. The Rockwell hardness number derives from the net increase in depth impression as the load on an indenter is increased from a fixed minor load to a major load and then returned to a minor load. Measurements are done on the R scale (minor load 10 kg; major load 60 kg; indenter 0.5 in×12.7 mm).

### Odor Measurement

The cast UV-cured plastic parts are qualitatively assessed for odor while cutting and grinding.

### Method for Making the Lens and UV-Curing

Lens compositions are degassed under vacuum, and cast into molds consisting of two glass plates and a plastic gasket. The molds are passed under a mercury UV lamp or other lamp at the desired wavelength, preferably in an inert atmosphere. The polymeric lenses thus obtained are annealed for 1 h at a temperature between 100° C. and 120° C. to eliminate residual stresses in the lenses before measurement of properties. In some cases a small piece of sheet-like polymer is obtained by cast polymerization and used to measure the refractive index and thermo-mechanical properties.

### Transparent Coatings or Films

UV-curable compositions are prepared by blending the components thereof. The mixture is degassed to remove air bubbles by application of vacuum with gentle heating, and applied on a desired surface using a variety of techniques, including draw down, spin coating, dip coating, forward and reverse roll coating, wire round rod coating, and die coating. The films are cured under a UV-lamp, or postbaked at high temperature.

### Characterization of the Coating or Film

For optical measurements about 1 μm thick transparent films are obtained from polymerizable mixtures which are drawn down to a film or spin coated onto a glass substrate using a Bird applicator, UV-cured by passing under a Hg UV-lamp and post-baked for 1 h at 100° C. In some cases a sheet-like polymer is obtained by cast polymerization. The cast or film-type polymers are measured for refractive index and evaluated for mechanical properties.

1. Monomers selected from the group consisting of the formulae (1), (2), (3) and mixtures thereof: 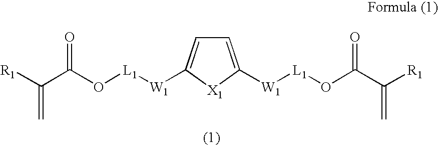
where
L₁ is defined as C₁-C₈ alkylene optionally interrupted by —S—, —SO₂—, —SO— and/or oxygen,
W₁ is a bond, sulfur or oxygen,
with the proviso that -L₁-W₁— or —W₁-L₁- contain at least one —S—, —SO₂— or —SO—,
X₁ is S, SO or SO₂,
and
R₁ is independently H or CH₃ 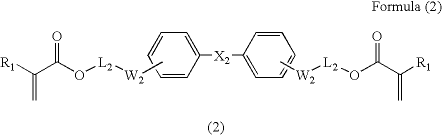
where
X₂ is a divalent linking group defined as a bond, —SO₂—, —SO—, —S—, —C(CH₃)₂—, —(CH₂)ₙ—S—(CH₂)ₙ—, —(CH₂)ₙ—SO—(CH₂)ₙ—, —(CH₂)ₙ—SO₂—(CH₂)ₙ—, —S—(CH₂)ₙ—S—, —SO—(CH₂)ₙ—SO— or —SO₂—(CH₂)ₙ—SO₂—,
and
W₂ is defined as a bond, sulfur, oxygen or a divalent linking group selected from the group consisting of —CONR₃—, —NR₃CO—, —SCONR₃—, —R₃NOCS—, —NR₃COS—, —SOCNR₃—, —CSO—, —OSC—, —COS—, —SOC—, —CSS—, —SSC—, —OCO—, —COO—, —SCOO—, —OOCS—, —OCONR₃— and —R₃NOCO—,
L₂ is C₁-C₁₀ alkylene which is optionally interrupted by W₂, —S—, —SO₂—, —SO— or oxygen,
with the proviso that at least one of -L₂-W₂— or —W₂-L₂- contain at least one of the divalent linking groups selected from the group consisting of —SCONR₃—, —R₃NOCS—, —NR₃COS—, —SOCNR₃—, —CSO—, —OSC—, —COS—, —SOC—, —CSS—, —SSC—, —SCOO—, and —OOCS—,
or
at least one of -L₂-W₂— or —W₂-L₂- contain —CONR₃—, —R₃NCO—, —OCONR₃—, —R₃NOCO—, —OCO—, —COO—, and at least one —S—, —SO₂— or —SO—,
or
-L₂-W₂— or —W₂-L₂- is a branched or linear C₁-C₄ alkylene substituted by OR₄ or SR₄,
R₃ is defined independently as H or CH₃,
R₄ is C₁-C₄ branched or linear alkyl or substituted or unsubstituted phenyl, and
R₁ is independently H or CH₃. 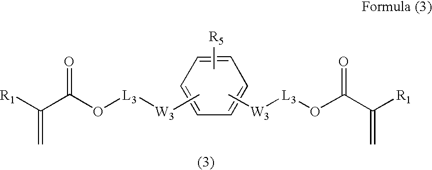
wherein
W₃ is a bond, —S—, —SO₂—, —SO— or a divalent linking group selected from the group consisting of —CONR₃—, —NR₃CO—, —SCONR₃—, —R₃NOCS—, —NR₃COS—, —SOCNR₃—, —CSO—, —OSC—, —COS—, —SOC—, —CSS—, —SSC—, —OCO—, —COO—, —SCOO—, —OOCS—, —OCONR₃— and —R₃NOCO—,
L₃ is C₁-C₁₀ alkylene which is optionally interrupted by W₃, —S—, —SO₂—, —SO— or oxygen,
with the proviso that at least one of -L₃-W₃— or —W₃-L₃- contain at least one of the divalent linking groups selected from the group consisting of —SCONR₃—, —R₃NOCS—, —NR₃COS—,
—SOCNR₃—, —CSO—, —OSC—, —COS—, —SOC—, —CSS—, —SSC—, —SCOO—, and —OOCS—,
or
at least one of -L₃-W₃— or —W₃-L₃- contain —CONR₃—, —R₃NCO—, —OCONR₃—, —R₃NOCO—, —OCO—, —COO—, and at least one —S—, —SO₂— or —SO—,
or
—W₃-L₃- or -L₃-W₃— is branched or linear C₁-C₄ alkylene substituted by OR₄ or SR₄,
R₃ is defined independently as H or CH₃,
R₄ is branched or linear C₁-C₄ alkyl or substituted or unsubstituted phenyl,
R₅ is hydrogen or branched or linear C₁-C₄ alkyl, and
R₁ is independently H or CH₃.

2. A high refractive index transparent plastic composition comprising of
a plastic formed from any one of the monomers according to claim 1,
optionally, a functionalized or surface treated nanoparticle,
and
optionally, at least one monomer selected from the group consisting of mono(meth)acrylate aromatic sulfur-containing monomers.

3. A plastic composition according to claim 2, wherein the nanoparticle is functionalized over at least a portion of its surface with a surface treatment agent so that the functionalized nanoparticle can copolymerize or react with the polymerizable resin during curing.

4. A plastic composition according to claim 2 containing a nanoparticle, wherein the nanoparticle is surface treated with agents selected from the group consisting of alcohols, amines, carboxylic acids, sulfonic acids, phosphonic acids, silanes and titanates.

5. A plastic composition according to claim 2 containing a nanoparticle, wherein the nanoparticle contains titanium, oxides of titanium, zirconium, oxides of zirconium, cerium, oxides of cerium or mixtures thereof.

6. A plastic composition according to claim 2, containing at least one mono(meth)acrylate aromatic sulfur-containing monomer.

7. A UV-cast optical lens formed from at least one of the monomers selected from the group consisting of formulae (1), (4), (5) and mixtures thereof, 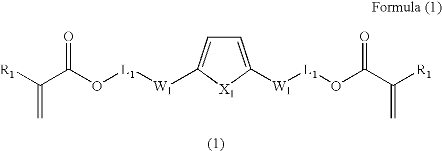
where
L₁ is defined C₁-C₆ alkylene optionally interrupted by sulfur and/or oxygen, W₁ is a bond, sulfur or oxygen with the proviso that at least one of -L₁-W₁— or —W₁-L₁- contain at least one —S—, —SO₂— or —SO—,
X₁ is S, SO or SO₂,
and
R₁ is independently H or CH₃, 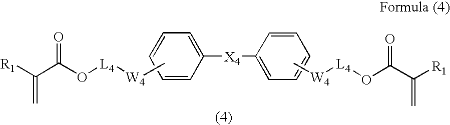
wherein
X₄ is a divalent linking group defined as —SO₂—, —SO—, —S—, —C(CH₃)₂—, —(CH₂)ₙ—S—(CH₂)ₙ—, —(CH₂)ₙ—SO—(CH₂)ₙ—, —(CH₂)ₙ—SO₂—(CH₂)ₙ—, —S—(CH₂)ₙ—S—, —SO—(CH₂)ₙ—SO— or —SO₂—(CH₂)ₙ—SO₂—,
n is 1-4,
W₄ is defined as a bond, sulfur or a divalent linking group selected from the group consisting of —OCO—, —COO—, —CO—, —SO—, —SO₂—, —OCOO—, —OOCO—, —CONR₃—, —NR₃CO—, —SCONR₃—, —R₃NOCS—, —NR₃COS—, —SOCNR₃—, —CSO—, —OSC—, —COS—, —SOC—, —OCO—, —COO—, —CSS—, —SSC—, —SCOO—, —OOCS—, —OCONR₃— and —R₃NOCO—,
L₄ is C₁-C₁₀ alkylene which is optionally interrupted by oxygen, —S—, —SO₂—, —SO—, oxygen or W₄
or L₄ is a branched or linear C₁-C₄ alkylene substituted by OH, OR₄ or SR₄,
R₃ is defined independently as H or CH₃,
R₄ is branched or linear C₁-C₄ alkyl or substituted or unsubstituted phenyl, R₁ is independently H or CH₃,
and 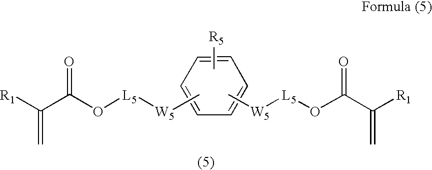
wherein
W₅ is a bond, oxygen of sulfur or a divalent linking group selected from the group consisting of —OCO—, —COO—, —CO—, —SO—, —SO₂—, —OCOO—, —OOCO—, —CONR₃—, —NR₃CO—, —SCONR₃—, —R₃NOCS—, —NR₃COS—, —SOCNR₃—, —CSO—, —OSC—, —COS—, —SOC—, —OCO—, —COO—, —CSS—, —SSC—, —SCOO—, —OOCS—, —OCONR₃— and —R₃NOCO—,
L₅ is C₁-C₁₀ alkylene optionally interrupted by oxygen, —S—, —SO₂—, —SO— or W₅,
or L₅ is a branched or linear C₁-C₄ alkylene substituted by OH, OR₄ or SR₄,
R₃ is defined independently as H or CH₃,
R₄ is branched or linear C₁-C₄ alkyl or substituted or unsubstituted phenyl,
R₅ is hydrogen or branched or linear C₁-C₄ alkyl and
R₁ is independently H or CH₃,
with the proviso that at least one of the -L₅-W₅— or —W₅-L₅- contains at least one sulfur.

8. A UV-cast lens according to claim 7 further comprising:
a functionalized or surface treated nanoparticle, wherein the surface treated or functionalized nanoparticle is functionalized over at least a portion of its surface with a surface treatment agent so that the particle can copolymerize or react with the polymerizable resin during curing.

9. A UV-cast lens according to claim 8, wherein the surface treated or functionalized nanoparticles contain zirconium, titanium or cerium.

10. A UV-cast lens according to claim 9, wherein the surface treated or functionalized nanoparticles are zirconium oxide, titanium oxide or cerium oxide.

11. A UV-cast lens according to claim 7, further comprising at least one monomer of the formula below 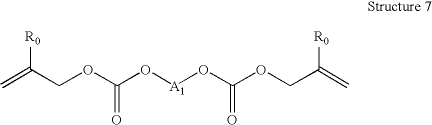
Where A₁ is a divalent dihydroxy aromatic-containing compound and each Ro is independently hydrogen, halo, or a C₁-C₄ alkyl group.

12. A UV-cast lens according to claim 7, further comprising at least one monomer of the formula below 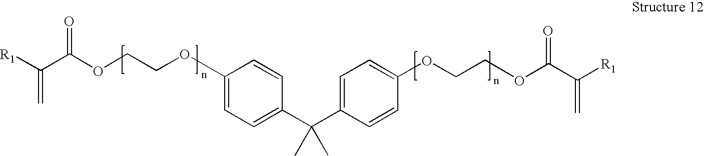
wherein n is 0 to 6 and RI is hydrogen or methyl.

13. The monomers defined as in Table 1:
TABLE 1
Specific High RI Monomers

| | |
|---|---|
| | 1 |
| 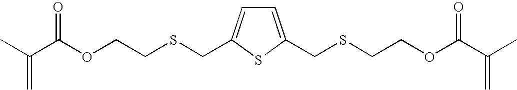 | |
| | 2 |
| 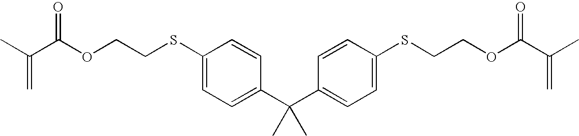 | |
| | 3 |
| 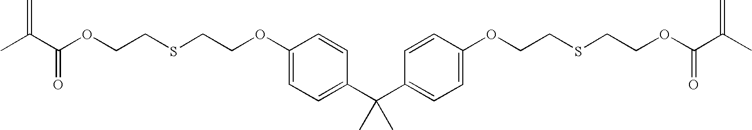 | |
| | 4 |
| 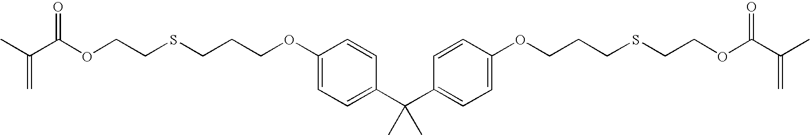 | |
| | 6 |
| 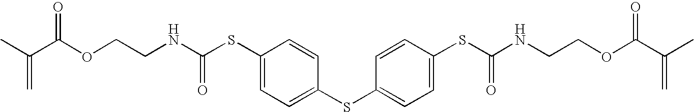 | |
| | 7 |
| 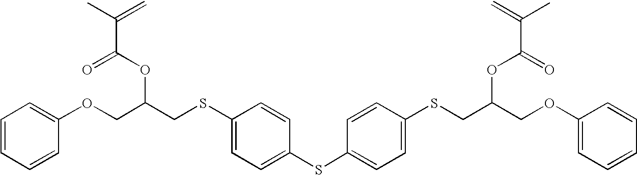 | |
| | 9 |
| 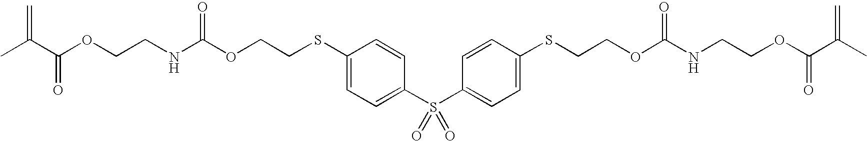 | |
| | 10 |
| 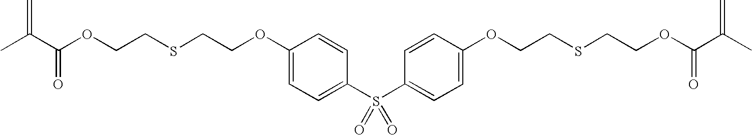 | |
| | 11 |
| 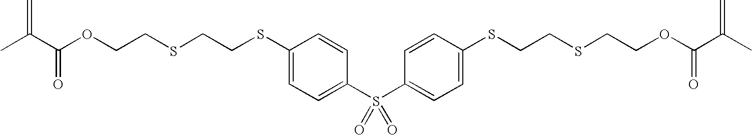 | |
| | 14 |
| 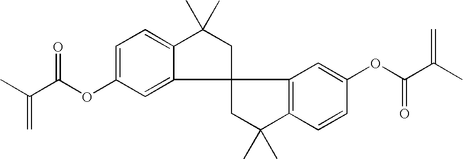 | |
| | 15 |
| 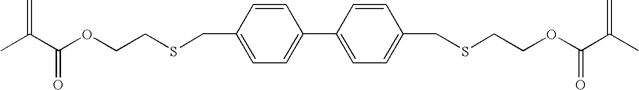 | |
| | 22 |
| 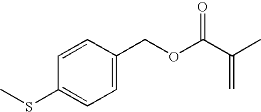 | |
| | 23 |
| 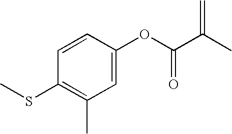 | |
| | 24 |
| 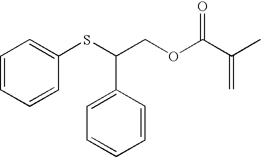 | |

14. A method of forming a high refractive index transparent material wherein the transparent material is a polymeric molded body, coating or film and the method comprises the steps:
placing a liquid composition into a mold cavity or assembly, wherein the mold assembly comprises a front mold member and a back mold member,
or
spreading the liquid composition onto a substrate to form a film or coating,
the liquid composition comprising at least one monomer selected from the group consisting of
formula (1), (2) and (3) according to claim 1,
optionally, a surface treated or functionalized nanoparticle,
and
a photoinitiator,
and
directing activating light toward at least one of the mold members or the film or coating to effect cure.

15. A method of forming a high refractive index polymeric eyeglass lens comprising the steps:
placing a liquid lens forming composition in a mold cavity or a mold assembly, wherein the mold assembly comprises a front mold member and a back mold member,
the lens forming composition comprising:
at least one monomer selected from the group consisting of formula (1), (4) and (5) according to claim 7,
optionally, a surface treated or functionalized nanoparticle,
and
a photoinitiator;
and
directing activating light toward at least one of the mold members subsequent to initiating cure of the lens to form the eyeglass lens.

16. A method of preparing bisthioethers of formulae (2′) and (3′) 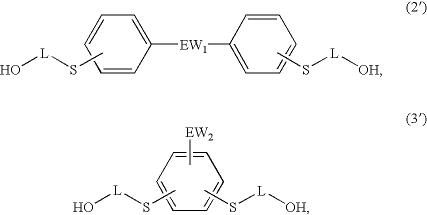
wherein L is C₂-C₆ alkyl or C₁-C₆ alkylene optionally interrupted with oxygen or sulfur and EW₁ and EW₂ are electron withdrawing groups,
by condensing a potassium salt of a hydroxyalkyl mercaptan with an aromatic halogen compound,
wherein the condensation takes place in a solvent selected from the group consisting of
dimethylformamide, dimethylacetamide, N,N-dimethylbutyramide, N,N-dibutylacetamide and N-methylpyrrolidinone.

17. A high refractive index transparent plastic composition comprising
a plastic formed from any one of the monomers according to claim 13,
optionally, a functionalized or surface treated nanoparticle,
and
optionally, at least one monomer selected from the group consisting of mono(meth)acrylate aromatic sulfur-containing monomers.

18. A plastic composition according to claim 17, wherein the mono(meth)acrylate aromatic sulfur-containing monomers are selected from the group consisting of

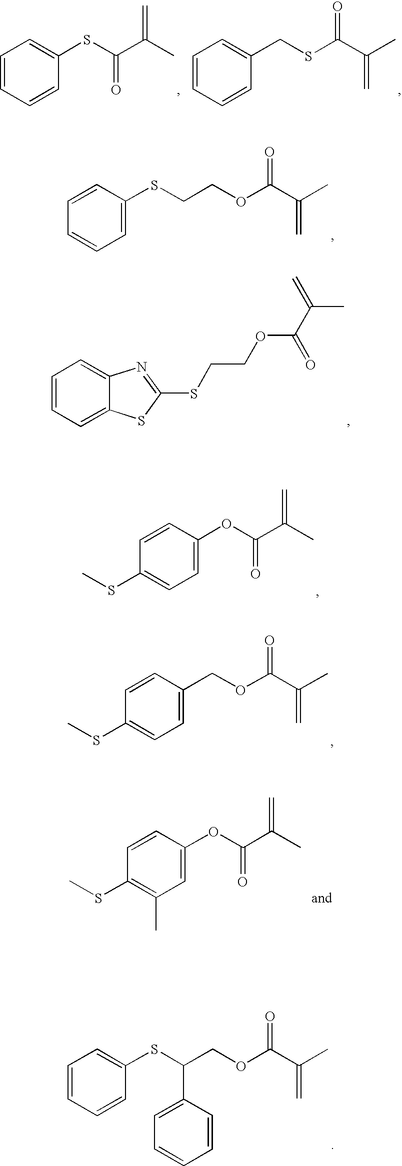

19. A plastic composition according to claim 17, wherein the composition is a polymeric molded body, coating or film.

20. A plastic composition according to claim 6, wherein the mono(meth)acrylate aromatic sulfur-containing monomers are selected from the group consisting of

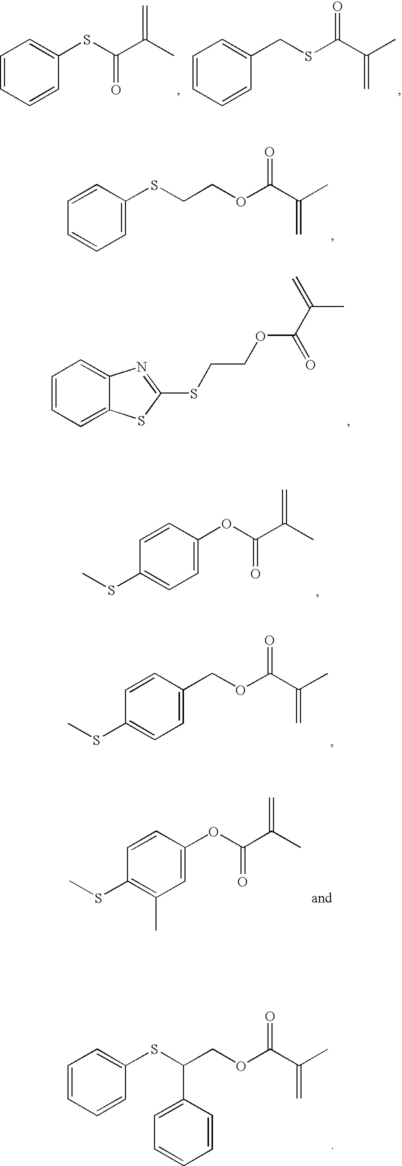

21. A UV-cast optical lens according to claim 7, which further contains at least one mono(meth)acrylate aromatic sulfur-containing monomer.

22. A UV-cast optical lens according to claim 21, wherein the mono(meth)acrylate aromatic sulfur-containing monomers are selected from the group consisting of

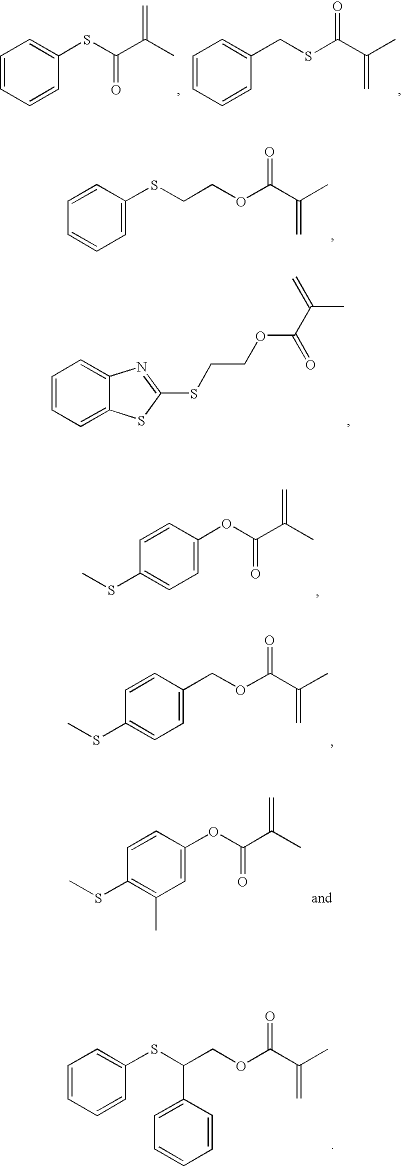

23. An ophthalmic lens, camera lens, visor, safety glasses, watch glasses, video disc, monitor, display, telecommunications systems, or medical/analytical equipment comprising the coating, film or polymeric molded body according to claim 19.

24. An ophthalmic lens, camera lens, visor, safety glasses, watch glasses, video disc, monitor, display, telecommunications systems, or medical/analytical equipment comprising the coating, film or polymeric molded body according to claim 2.

25. A method of forming a high refractive index transparent material wherein the transparent material is a polymeric molded body, coating or film and the method comprises the steps:
placing a liquid composition into a mold cavity or assembly, wherein the mold assembly comprises a front mold member and a back mold member,
or
spreading the liquid composition onto a substrate to form a film or coating,
the liquid composition comprising at least one monomer selected from the monomers according to claim 13,
optionally, a surface treated or functionalized nanoparticle,
and
a photoinitiator,
and
directing activating light toward at least one of the mold members or the film or coating to effect cure.

26. A method according to claim 25, wherein the liquid composition further comprises a mono(meth)acrylate aromatic sulfur-containing monomer.

27. A method according to claim 26, wherein the mono(meth)acrylate aromatic sulfur-containing monomer is selected from the group consisting of

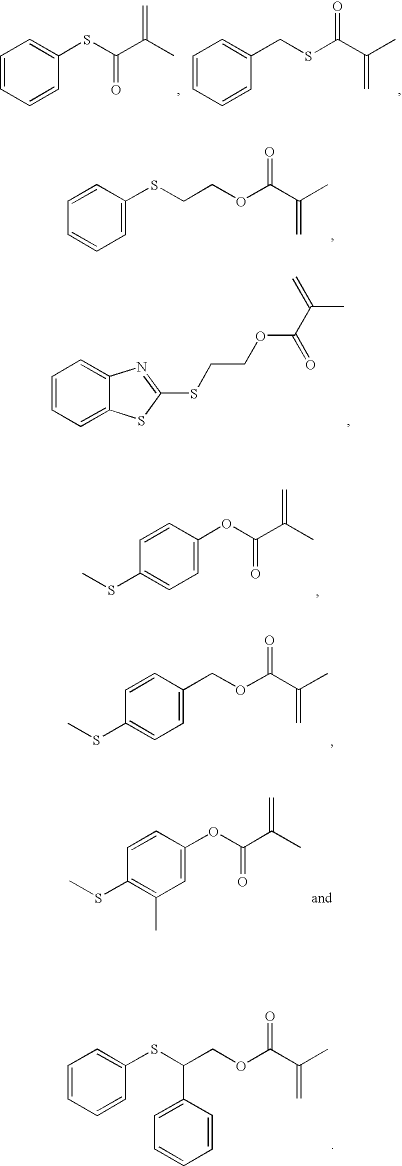

28. A method according to claim 14, wherein the liquid composition further comprises a mono(meth)acrylate aromatic sulfur-containing monomer.

29. A method according to claim 15, wherein the lens forming composition further comprises a mono(meth)acrylate aromatic sulfur-containing monomer.

30. A method according to claim 29, wherein the mono(meth)acrylate aromatic sulfur-containing monomer is selected from the group consisting of [chemical structure]
